(19) United States
(12) Patent Application Publication
(10) Pub. No.: US 2026/0119552 A1
(43) Pub. Date: Apr. 30, 2026

(54) SEARCHING OVER UNSTRUCTURED RECORDS UTILIZING A TAILORED DOMAIN RETRIEVAL-AUGMENTED GENERATION

(71) Applicant: Oracle International Corporation, Redwood Shores, CA (US)

(72) Inventors: Daniela Weiss, Los Angeles, CA (US); Charles Woodrow Dickstein, New York, NY (US); Devashish Khatwani, Seattle, WA (US); Srinivasa Phani Kumar Gadde, Fremont, CA (US); Vishal Vishnoi, Redwood City, CA (US)

(73) Assignee: Oracle International Corporation, Redwood Shores, CA (US)

(21) Appl. No.: 19/368,190

(22) Filed: Oct. 24, 2025

Related U.S. Application Data
(60) Provisional application No. 63/712,371, filed on Oct. 25, 2024.

(51) Int. Cl. G06F 16/334; G16H 10/60

(52) U.S. Cl. CPC G06F 16/3344; G16H 10/60

(57) ABSTRACT

A computer-implemented method includes receiving a query in natural language, generating an input for a large language model, the input including a prompt generated based on the query, and identifying a plurality of slots associated with a plurality of sections of a content item. The method further includes generating a query result based on the input, the query result including a subset of the plurality of slots selected, extracting one or more document chunks from a database storing a plurality of document chunks as one or more relevant document chunks associated with the query result, formatting the relevant document chunks into a response to the query, and providing the response to a client system. The plurality of document chunks is generated by dividing each content item of a plurality of content items into the plurality of document chunks based on sections within each content item.

### CROSS-REFERENCE TO RELATED APPLICATIONS

The present application is a non-provisional application of and claims the benefit and priority to U.S. Provisional Application No. 63/712,371, filed Oct. 25, 2024, the entire contents of which is incorporated herein by reference for all purposes.

### BACKGROUND

Healthcare providers typically perform several common tasks for each patient repetitively. As an example, regardless of the complexity of a condition or the number of interactions for the same condition, a healthcare provider typically records the interaction in a medical note. These notes typically record information as data that lacks predefined formats and structure making it hard to organize, search, and analyze. Due to the volume and unstructured nature of this data, it can be difficult to retrieve relevant pieces of information to resolve a received question or query. Therefore, it may be desirable to provide improved techniques for searching over unstructured records.

### BRIEF SUMMARY

Techniques disclosed herein pertain to generative artificial intelligence (AI) systems, and, more specifically, to search mechanisms for agentic AI systems.

In embodiments, a computer-implemented method includes receiving, by a computer system, a query in natural language and generating, by the computer system, an input for a generative large language model, the input comprising a prompt generated based on the query. The method further includes identifying, by the computer system, a plurality of slots associated with a plurality of sections of a content item, generating, by the generative large language model, a query result based on the input, the query result comprising a subset of slots of the plurality of slots selected in accordance with the plurality of sections, extracting, by the generative large language model, one or more document chunks from a database storing a plurality of document chunks as one or more relevant document chunks associated with the query result, formatting, by the computer system, the one or more relevant document chunks into a response to the query, and providing, by the computer system, the response to a client system. The plurality of document chunks is generated by dividing each content item of a plurality of content items into the plurality of document chunks based on sections within each content item of the plurality of content items.

In certain embodiments, the plurality of slots includes at least one of medical named entity recognitions (NERs), keywords identified as frequently arising in clinical patient encounters, questions identified as frequently arising in clinical patient encounters, and information identified as frequently requested during clinical patient encounters. In embodiments, the document chunks in the database are produced by performing at least one of sliding window chunking and semantic chunking.

In certain embodiments, extracting one or more document chunks from the database storing the plurality of document chunks further comprises extracting metadata associated with the one or more relevant document chunks. In embodiments, the metadata comprises at least one of a date of creation of a note, a note type, a note section, a specialist note, patient information, and a practitioner role. In embodiments, extracting the one or more document chunks from the database comprises using the metadata to determine a relevance of the one or more of the plurality of document chunks into the query.

In certain embodiments, extracting one or more document chunks from the database further includes identifying a time-dependent aspect in the query, and selecting one or more of the plurality of document chunks based on the time-dependent aspect in the query.

In certain embodiments, extracting one or more document chunks from the database further comprises performing at least one of a keyword search and a k-Nearest Neighbor (KNN) search on the plurality of document chunks in the database.

In certain embodiments, the method further includes enriching, by the computer system, at least one of the plurality of sections and the plurality of document chunks with embeddings. In embodiments, the embeddings include at least one of subsection header names and vector embeddings.

In certain embodiments, formatting the one or more relevant document chunks into the response to the query comprises ranking the one or more relevant document chunks based on a predefined set of parameters.

In embodiments, a system includes a computer system comprising one or more processors and memory storing computer executable instructions that, when executed by the one or more processors, configure the computer system to at least receive a query in natural language, generate an input for a generative large language model, the input comprising a prompt generated based on the query, identify a plurality of slots associated with a plurality of sections of a content item, generating, by the generative large language model, a query result based on the input, the query result comprising a subset of slots of the plurality of slots selected in accordance with the plurality of sections, extract, by the generative large language model one or more document chunks from a database storing a plurality of document chunks as one or more relevant document chunks associated with the query result, format the one or more relevant document chunks into a response to the query, and provide the response to the query to a client system. The plurality of document chunks are generated by dividing each document of a plurality of documents into the plurality of document chunks based on sections within each content item of the plurality of content items.

In embodiments, the plurality of slots comprises at least one of medical named entity recognitions (NERs), keywords identified as frequently arising in clinical patient encounters, questions identified as frequently arising in clinical patient encounters, and information identified as frequently requested during clinical patient encounters.

In certain embodiments, the document chunks in the database are produced by performing at least one of sliding window chunking and semantic chunking.

In certain embodiments, extracting one or more document chunks from the database storing the plurality of document chunks further comprises extracting metadata associated with the one or more relevant document chunks. In embodiments, the metadata comprises at least one of a date of creation of a note, a note type, a note section, a specialist note, patient information, and a practitioner role. In embodiments, extracting the one or more document chunks from the database comprises using the metadata to determine a relevance of the one or more of the plurality of document chunks into the query.

In certain embodiments, extracting one or more document chunks from the database further includes identifying a time-dependent aspect in the query, and selecting one or more of the plurality of document chunks based on the time-dependent aspects in the query.

In embodiments, extracting one or more document chunks from the database further comprises performing at least one of a keyword search and a k-Nearest Neighbor (KNN) search on the plurality of document chunks in the database.

In embodiments, a non-transitory computer-readable medium storing computer-executable instructions that, when executed by one or more processors of a computer system, configure the computer system to at least receive a query in natural language, generate an input for a generative large language model, the input comprising a prompt generated based on the query, identify a plurality of slots associated with a plurality of sections of a content item, generate, by the large language model, a query result based on the input, the query result comprising a subset of slots of the plurality of slots selected in accordance with the plurality of sections, extract, by the generative large language model, one or more document chunks from a database storing a plurality of document chunks as one or more relevant document chunks associated with the query result, format the one or more relevant document chunks into a response to the query, and providing the response to the query to a client system. The plurality of document chunks are generated by dividing each document of a plurality of documents into the plurality of document chunks based on sections within each content item of the plurality of content items.

As used herein, a section encompasses a logical division of a content item that reflects a human-recognizable topical or structural boundary within the item. Example of sections included standard clinical sections (e.g., Chief Complaint, History of Present Illness, Review of Systems, Physical Exam, Assessment & Plan), headings, subheadings, templated blocks, or other labeled/unlabeled text regions that function as a coherent unit. Sections may be detected using explicit headers, templates, rules, dictionaries, machine-learned classifiers, or combinations thereof. Where no explicit headers exist, contiguous text regions having coherent topic or function are treated as sections. Sections provide a document-level structure used for ingestion, indexing, filtering, ranking, and presentation.

As used herein, a slot encompasses a predefined or inferred information target associated with a section type and/or use case, representing a semantic category or query-relevant facet expected to occur in a content item. Slots include named entities (e.g., problem, medication, allergy), section-specific concepts (e.g., “chief complaint statement,” “assessment diagnosis,” “plan instructions”), frequently asked questions, keywords, or other information needs curated for the domain. Slots may be (i) curated schema elements and dictionaries, (ii) dynamically predicted for a given query via a planner/LLM, or (iii) both (curated base set refined by prediction). A slot can be satisfied by one or more chunks; a single chunk may satisfy multiple slots. Slots guide selection, filtering, and ranking of chunks within and across sections. Slot predictions constrain the retrieval search space, inform metadata filters (e.g., note type, time window), and influence ranking and response formatting.

As used herein, a chunk encompasses a contiguous or near-contiguous unit of content derived from a content item for storage, indexing, retrieval, or ranking. A chunk may be (i) an entire section, (ii) a subsection of a section, or (iii) a windowed or semantically segmented span when section boundaries are absent or unreliable. Chunks may be produced using sliding windows (with configurable size/stride), semantic segmentation, header-based splitting, or hybrids thereof. Each chunk may carry associated embeddings, identifiers, scores, and metadata (e.g., note type, section label, author, date, patient, practitioner role). Chunks are the primary retrieval units used by keyword, vector (e.g., KNN), hybrid, and metadata-filtered search; they are ranked and assembled into responses.

Unless otherwise clear in context, a chunk may coincide with an entire section or a subsection thereof, and a slot may be satisfied by one or more chunks. In some embodiments, slots are curated and/or predicted per-query, and chunks carry embeddings and metadata enabling keyword, vector, hybrid, and temporally constrained retrieval.

The techniques described above and below may be implemented in a number of ways and in a number of contexts. Several example implementations and contexts are provided with reference to the following figures, as described below in more detail. However, the following implementations and contexts are but a few of many.

### BRIEF DESCRIPTION OF THE DRAWINGS

The present disclosure is described in conjunction with the appended figures, where like components are indicated with like reference numbers.

FIG. 1 is a block diagram illustrating an example computing environment, in accordance with embodiments.

FIG. 2 is a block diagram illustrating an example of the function of the extractor of FIG. 1, in accordance with embodiments.

FIG. 3 is an example of a response generated by computing environment of FIG. 1, in accordance with embodiments.

FIG. 4 is a flowchart illustrating an example process for generating a response in accordance with a query, in accordance with embodiments.

FIGS. 5 and 6 are flowcharts illustrating an example process for generating a query, in accordance with embodiments.

FIG. 7 is a block diagram illustrating an example computing environment incorporating agent-driven services, according to certain environments.

FIG. 8 is a block diagram illustrating an alternative example computing environment for automatically generating knowledge-grounded response data, according to certain embodiments.

FIG. 9 is a block diagram illustrating another example computing environment incorporating an agent-driven digital assistant system for automatically generating knowledge-grounded response data, according to certain embodiments.

FIG. 10 is a block diagram illustrating one pattern for implementing a cloud infrastructure as a service system, according to at least one embodiment.

FIG. 11 is a block diagram illustrating another pattern for implementing a cloud infrastructure as a service system, according to at least one embodiment.

FIG. 12 is a block diagram illustrating another pattern for implementing a cloud infrastructure as a service system, according to at least one embodiment.

FIG. 13 is a block diagram illustrating another pattern for implementing a cloud infrastructure as a service system, according to at least one embodiment.

FIG. 14 is a block diagram illustrating an example computer system, according to at least one embodiment.

### DETAILED DESCRIPTION

In the following description, for the purposes of explanation, specific details are set forth in order to provide a thorough understanding of certain embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

### Introduction

A traditional activity performed by healthcare providers is the generation of a variety of notes and records related to patient interactions, medications, patient observations, diagnoses, and other medical data. Traditionally, these notes and records are stored as unstructured data in a storage location such as an electronic health record (EHR). For instance, freeform entries in text fields in a patient record are not structured in specific ways with syntax rules. Such unstructured data lack predefined formats and structure, which make them difficult to organize, search, and analyze. In particular, in the medical industry where medical providers are frequently making notes about patient encounters, it can be challenging to retrieve information relevant to a query if these notes are stored in various unstructured formats. Patient history records often include decades worth of information, much of which have been scanned in or otherwise processed from original paper records into the EHR. While optical character recognition or similar data processing may have converted such records into a searchable format and these unstructured data records lack the document structure, logical rules, metadata, and tags to facilitate efficient extraction of relevant portions of those records in response to any query, even with the assistance of modern tools such as artificial intelligence.

The lack of an efficient and reliable way to search unstructured text while enabling the generation of accurate responses to the search query is a significant pain point for a medical professional. Even with an EHR on hand, medical clinicians must dedicate significant amount of time reviewing information in medical charts, particularly combing through unstructured data in the patient histories. While an EHR allows storage of a large amount of information, such as decades worth of patient records, the manner in which such information is stored makes retrieval of the data in those records slow, unreliable, and inaccurate.

It would be desirable to efficiently extract relevant clinical information in a refined summary format, even including data drawn from unstructured data sources, as such a process would result in a significant increase in the ability of a medical professional to provide quality, informed patient care. Being able to extract relevant clinical information accurately, out of structured and unstructured data stores, in a concise format based on a combination of logical rules, artificial intelligence, and document structure would be particularly valuable. Further, it would be highly desirable to be able to surface relevant information based on the initial query, contextual information, patient history, and other factors influencing the relevancy of particular pieces of information out of a large amount of unstructured data.

A common way to extract relevant information from an external knowledge base (such as an EHR) is to use a Retrieval-Augmented Generation (RAG) approach, which enables finding relevant information from an external knowledge base to improve the responses generated by a large language model (LLM) by providing more context for the query received at the LLM. For instance, in response to a query received from a user, a RAG system converts this query into a vector representation, or embedding, to search an external knowledge base for “chunks” that are semantically similar to the query's embedding. The retrieved information may be ranked and prioritized before being made available to the LLM. By using the ranked, prioritized information as context, the LLM is able to generate a more accurate response to the initial query.

However, the traditional RAG system is still problematic if the external knowledge base includes unstructured data without semantic classification or metadata.

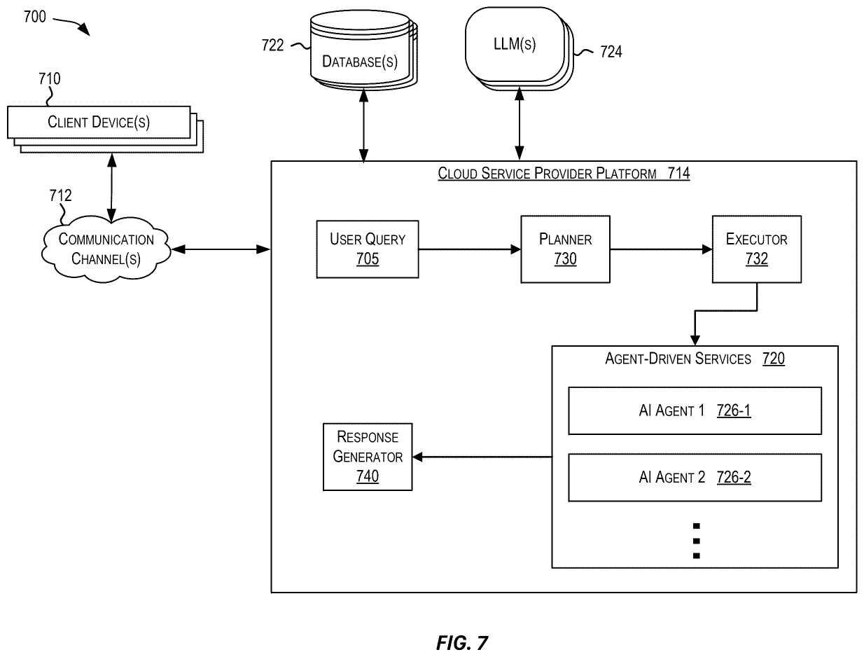

Instead, the present disclosure provides a tailored RAG system with additional steps to pre-process (i.e., ingest) the unstructured data, refine the semantic search over the preprocessed data, and pre-process the initial query (i.e., provide an improved “query understanding”) to anticipate the portions of the preprocessed data most likely to be relevant to the query. Such a tailored RAG system may be implemented, for example, as one of the agents included as part of a computing environment incorporating agent-driven services, such as discussed with respect to FIG. 7-9 at appropriate junctures hereinafter. As will be discussed in further detail below, the present disclosure provides:
Searching of unstructured patient records, which have been chunked to retrieve relevant portions of content items (e.g., clinical notes) based on a curated similarity score between the query and available document chunk embeddings;
A tailored clinical domain RAG system using a hybrid-based semantic search over the unstructured content items to find the relevant unstructured piece of text within the content items in response to the query.
Processing of the initial query to predict the likely relevant slots (e.g., note types, specific encounters, time periods, indications, etc.) to prioritize the relevant slots in the retrieving of chunks.

In this way, by providing a query understanding stage to produce both a refined input to suitable for the LLM as well as a curated set of data from which the LLM draws the data used in generating the query response, the present technique enables an improvement over the traditional RAG system. While the present disclosure mentions the use of LLMs as an example mechanism for analyzing data and generating summary reports, it is noted that other artificial intelligence techniques may be used including, and not limited to, Small Language Models (SLMs), Multi-modal Models, reasoning models and chain-of-thought architectures, transformer-based models.

Further, in embodiments, a combination of vector database queries may be used to finalize a semantic score, taking into account a variety of factors such as metadata filters and the temporality of the documents (e.g., more recently generated notes or notes mentioning specific concerns are prioritized over routine notes). Additionally, such consideration of unstructured data may be combined and crosschecked with existing structured data in the EHR to further refine the accuracy of the results produced by the overall system.

### Overview

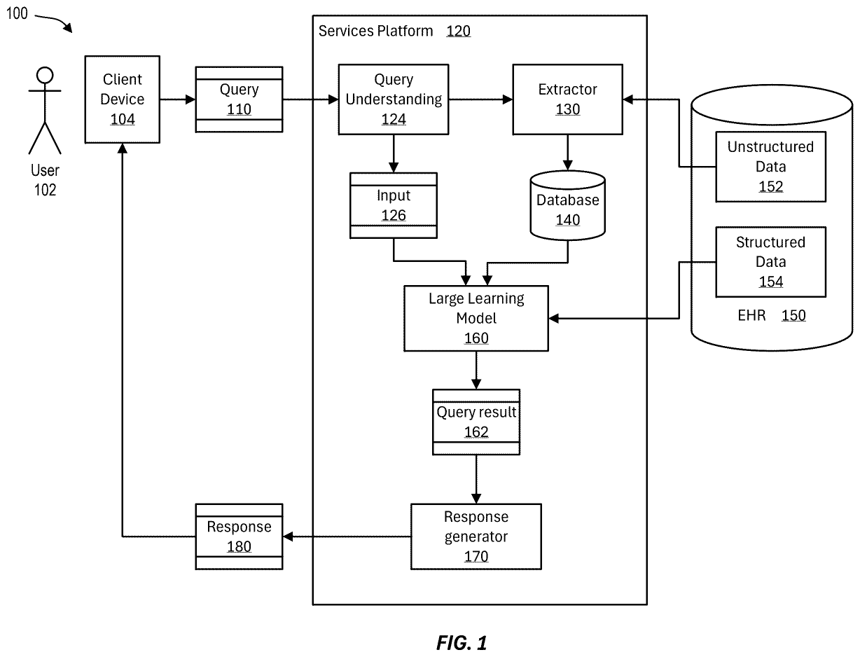

Referring now to FIG. 1, a computing environment 100 includes a user 102 operating a client device 104 to generate a query 110. Query 110 may be generated, for instance, in response to a question provided by the user in natural language (e.g., typed query, verbal input, voice-to-text entry using conversational language), structured commands entered by text or voice, selection of specific options at a user interface at the client device, and other ways of providing the basis of a query. For example, the user may be a medical professional providing a query including a question regarding a particular condition of a specific patient, a general medical history summary of a patient with an upcoming appointment with the user.

While query 110 is shown as originating from client device 104, it is noted that query 110 may be initiated by a user input (e.g., a request for information, a natural language voice input, selection at a user interface, etc.) at a client device, or transmitted information from a computer system (e.g., a command from an application with access to the EHR and/or client device, a different portion of an automated system, an automated command from an appointment management system in response to an upcoming appointment reminder, an automated agent within a service provider platform, etc.). The query may contain, for example, information regarding relevant dates, patient identification, authentication status, specific medical conditions, insurance information, and other information that may pertain to the data to be retrieved in subsequent steps. For instance, the query may have been generated by an agentic artificial intelligence (agentic AI) in response to a different query received at the agentic AI, then passed to services platform 120 to be processed therein, where a plurality of such automated agents form an agentic AI system.

Query 110 is passed to a services platform 120. At services platform 120, the query is received by a query understanding stage 124. In embodiments, the query may first be routed by a planner (as will be discussed at an appropriate point below).

At query understanding stage 124, the query is processed using a predefined set of parameters to extract contextual information about the query, such as identifying information about the patient, dates of recent visits with the user, recently created records in the EHR within a predefined time period (e.g., last month, last 6-months, etc.), a specific condition or diagnosis, and other information that may be used to further narrow the search based on the query. The predefined set of parameters may also include, for example, information known as being frequently required for medical providers. The parameters may also include enrichments, if any, added to the document records at ingestion time (e.g., when the original notes were entered into the EHR) such as types of notes, rules and guidelines that predict certain sets of clinical note type sets, questions that frequently come up for medical providers, and other factors. For instance, in the query understanding stage, a query from a user, a user device, a computing system, an automated agent, or other input source may be augmented with context data, such as patient identification, physician identification, current date and time, information regarding a specific workspace (e.g., patient chart and/or medical code), previous related inquiries, and others.

Query understanding stage 124 may also generate predictions of relevant chunks or slots of information, based on the contextual information extracted from the query, thus using the context information to help narrow down the search space, especially of unstructured documents within the EHR, for example. As an example, query understanding stage 124 may implement an automated planner function to generate predictions of candidate actions to be performed in response to the query. The automated planner function may then generate specific metes and bounds of actions to be taken by a subsequent large language model, for instance, including start and end date, relevant practitioner identification, types and sections of notes to be evaluated, types of medical entities relevant to the query, and preset limits and sorting parameters. The results of the processing at the query understanding stage results in an input 126 suitable for feeding into a large language model (LLM). Similarly, the same results from the query understanding stage may be fed into an extractor 130, which functions to extract the likely relevant chunks or slots of information into a database 140, such as a vector database providing an optimized search space for a downstream LLM.

In an embodiment, extractor 130 uses the results of query understanding stage 124 to extract the likely relevant data from an EHR 150, which includes both unstructured data 152 and structured data 154. Extractor 130 may use the context information from query understanding stage 124 to ingest components of the unstructured data into document chunks, for instance, in a fast healthcare interoperability resources (FHIR) format, a semantic object (SO) format, etc. Extractor 130 may also extract nested information, such as document titles, note types, reformatting dates, and other information embedded in specific content items. The content items, such as clinical notes, may be enriched during this stage using domain specific information.

Extractor 130 may further extract specific chunks based on various rules, including the parameters used in the query understanding. For instance, standard clinical sections from notes may be extracted and chunked into smaller chunks or slots. The sections may also be enriched with subsection header names and/or vector embeddings. The chunks content and embedding may additionally be stored in an index with metadata properties such as note type, note title, section, patient, physician, etc. Further, within the information provided from the query understanding stage, relevant medical concepts named entity recognitions (NER), such as problems and medications can be extracted in addition to meta data related to unstructured clinical notes (sections, note types). Further, in addition to clinical entities, additional values such as logical temporal periods for which the search space should be scoped may be extracted. Such information aid in the implementation of post processing logic and providing further contextual information for the LLM based on the inferred temporality of the query. In embodiments, extractor 130 may use a combination of a similarity score based search query, such as a hybrid search (e.g., a keyword search plus a K-Nearest Neighbor (KNN) search) and a combination of filters that are processed with additional logic to match the subset of documents in database 140, to be called on the query against embedded document chunks. The K most semantically relevant unstructured document chunks in clinical notes may be retrieved in order to resolve the question. In certain embodiments, the extracted data may also be prioritized and ranked according to factors such as relevancy scores, a decay score (e.g., portions of notes created within the past month, the past 6-months, etc., are ranked higher than older information), specific conditions mentioned in the note, and others.

Input 126 is provided to a LLM 160, which is also given access to database 140 as a curated data source for retrieval of the appropriate information in generating a response to the query. It is noted that LLM 160 may also pull information from structured data stored within EHR 150, such as enriched data with standardized embeddings, which are more readily searched by LLM 160. LLM 160 then generates a query result 162, which is provided to a response generator 170. Query result 162 may include a subset of slots or chunks from database 140 as deemed by LLM 160 to be relevant and suitable for use as part of a response to be generated by the system. Response generator 170 then formats the query result, including the subset of slots or chunks, into a standard or user-specified format to generate a response 180, which is then provided as output at client device 104.

Regarding the ingestion of unstructured data, it is recognized herein that clinical notes generally have inherent semantic structure and standard clinical sections, such as the SOAP format disclosed herein. Also, it is recognized herein that queries often relate to specific content found within sections of documents, such as a patient's perceived demeanor in the Subjective section of a SOAP note.

Given these recognitions, the presently disclosed approaches uses a multi-layer chunking method. Initially, the unstructured documents may be chunked at the section layer, which enables the extraction of entire sections of content items in the response to the user, without missing content, if appropriate. That is, it is recognized herein that, for specific contexts, query responses often require presentation of whole sections from a content item.

### Query and Data Ingestion

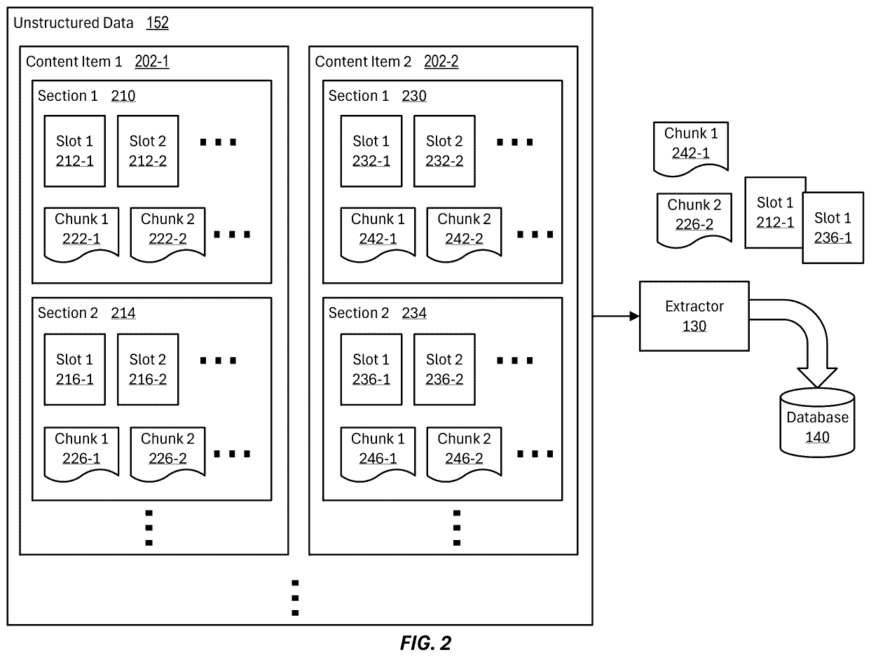

FIG. 2 shows a block diagram illustrating an example ingestion process, in accordance with embodiments. As shown in FIG. 2, unstructured data 152 may include a plurality of content items (e.g., content item 1 (202-1), content item 2 (202-2), and so on, as indicated by ellipsis). For example, a content item may be a discrete, self-contained unit of digital information that can be individually stored, managed, and retrieved within a computer system, such as EHR 150. That is, a content item may represent any distinct piece of content, such as a document, image, video, web page, database record, or social media post, or portion thereof, that exists as an independent entity potentially with its own unique identifier and associated metadata (like creation date, author, and permissions). Each content item may include a plurality of sections, such as section 1 (210), section 2 (214) within content item 1 (202-1), and section 1 (230), section 2 (234) within content item 2 (202-2), and so on. Further, within each section, there may be a plurality of identified slots (e.g., slot 1 (212-1), slot 2 (212-2) within section 1 (210) of content item 1 (202-1)) and document chunks (e.g., chunk 1 (222-1), chunk 2 (222-2) within section 1 (210) of content item 1 (202-1)).

The slots and chunks within each section may have been pre-identified or newly identified by extractor 130 for a given query. For example, each slot or chunk may include medical named entity recognitions (NERs), keywords identified as frequently arising in clinical patient encounters, questions identified as frequently arising in clinical patient encounters, and information identified as frequently requested during clinical patient encounters. The frequency may be defined, for example, based on statistical information using historical data of patient visits at a particular clinic, geographical area, within a certain period of time, or other metrics.

While the slots and chunks are shown as separate items in FIG. 2, a slot may form a portion of a chunk, a slot may include multiple chunks, and a chunk may contain multiple slots, depending on the granularity required in classifying portions of the section within the content item. For instance, sections may be identified using a set of rules and common identifiers using curated clinical dictionaries. Or, as noted above, an entire section may be chunked as a single chunk, in order to enable providing an entire section as a query result if so warranted. Document chunks may be further chunked when a section is not identified, to ensure all documents are ‘searchable’ and embedded. While different note types may contain different structures, certain standard clinical sections are commonly used and frequently appear in any note type. Such structures include, and are not limited to, a Chief Complaint (CC), History of Present Illness (HPI), Review of Systems (ROS), Physical Exam (PE), Assessment and Plan (A&P), labs, problem list, medication list, patient education, social history, family history, and other common categories of topics. The chunking may be performed using a variety of methods such as sliding window chunk, semantic chunking, or a combination thereof.

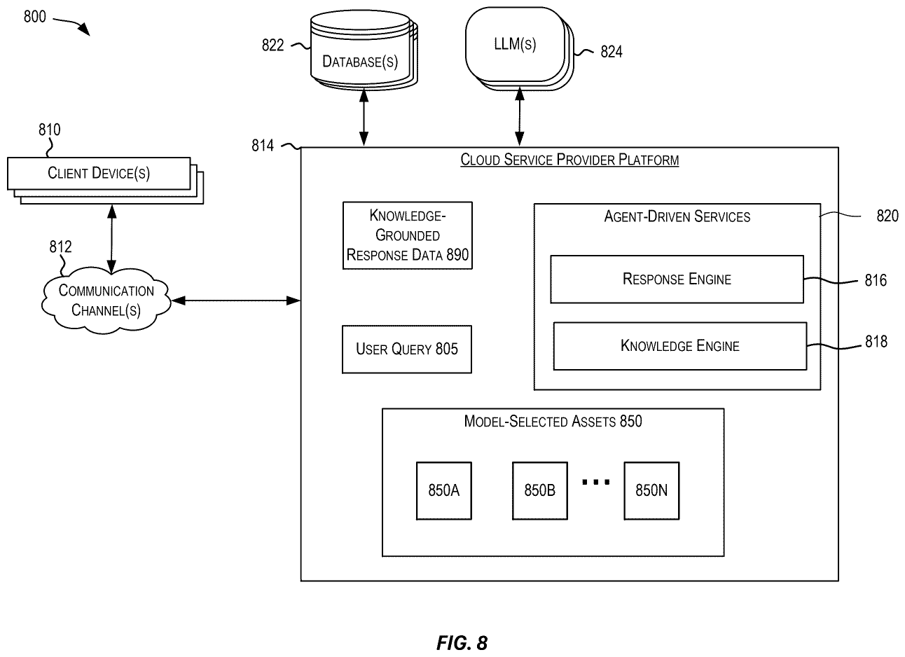

Based on the information provided by the query understanding stage, extractor 130 extracts specific combinations of chunks and/or slots and provides them to database 140 as a curated basis from which derive information for use by the LLM. In examples, database 140 may be considered an implementation of knowledge-grounded response data 890, as described below with respect to FIG. 8. As shown in FIG. 1, LLM 160 provides query result 162, which is processed and formatted by response generator 170 to generate response 180.

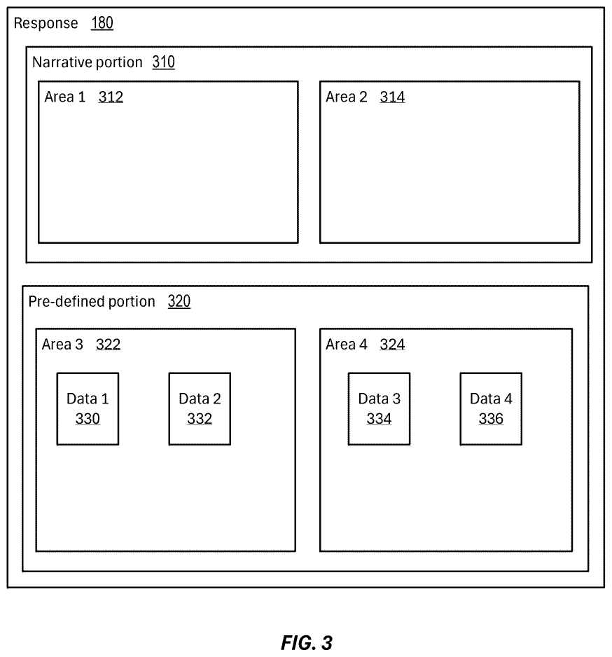

An example of a format for response 180 is shown in FIG. 3. As shown in FIG. 3, response 180 may include a narrative portion 310 and a pre-defined portion 320. In embodiments, narrative portion 310 may include a summary of patient information in a narrative format, generated by LLM 160 and/or quoted from selected slots, chunks, or sections of content items, as shown in FIG. 2. For example, an area 1 (312) of narrative portion 310 may include a generated summary of the current patient information, such as recent history of clinical visits by the patient, and recent diagnostic results related to the Chief Complaint. An area 2 (314) may include a narrative summary of the patient's medication history and family history, for example.

Pre-defined portion 320 may include extracted information from the structured data portion of the EHR, formatted in a standardized manner, such as the patient's vitals at the visit, active prescriptions, recent laboratory results, form fields to enter additional notes by the medical clinician, and other data. For instance, an area 3 (322) may include data 1 (330), such as patient's current vitals, data 2 (332) such as a list of active prescriptions, presented in a list format. An area 4 (324) may include data 3 (334), such as a list of recent clinic visits and chief complaints, and data 4 (336) as a graph of recent blood test results as related to the current chief complaint. Other formats of the response presentation may be defined for specific users and/or use case scenarios.

### Illustrative Methods

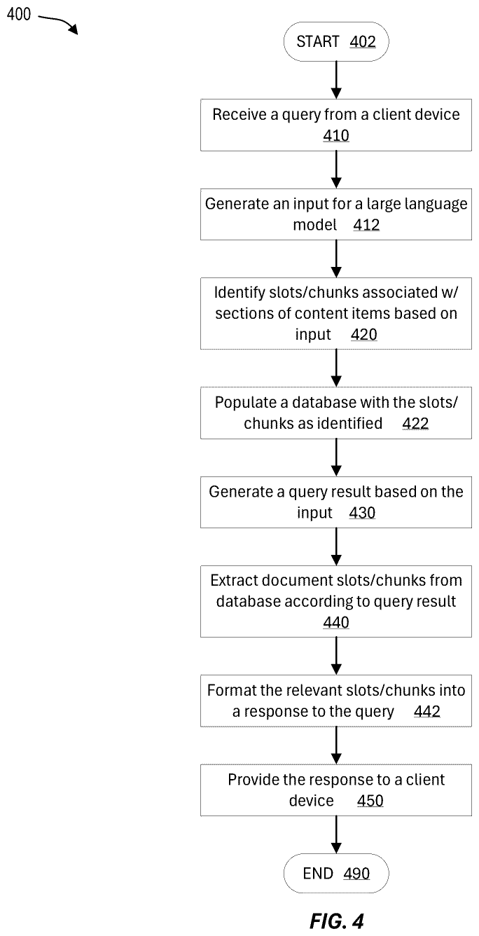

FIG. 4 shows a flowchart illustrating an example process of implementing the computing environment of FIG. 1. As shown in FIG. 4, a process 400 begins with a start step 402 and proceeds to receive a query from a client device in 410. As discussed above, the query may be generated by the client device in response to a user providing text, voice, or other input at the client device. In examples, the user input may take the form of a natural language text input by the user as text or via a voice-to-text input interface.

Process 400 proceeds to generate an input for a large language model in 412. Such a step may be performed, for instance, using query understanding stage 124 as shown in FIG. 1.

Based on the input, process 400 proceeds to identify, in 420, one or more slots/chunks associated with section of content items, as may be performed by extractor 130 of FIG. 1 or FIG. 2. The slots and/or chunks so identified are used to populate a database, such as database 140 of FIGS. 1 and 3, in block 422.

Also, based on the input, a query result is produced by, for example, a LLM in 430. The query result may include, for example, a subset of slots/chunks identified in 420 and stored in 422. Based on the query result, a specific subset of slots and/or chunks are extracted from the database in 440 (e.g., by response generator 170 of FIG. 1), then formatted into a response in 442 (e.g., as shown in FIG. 3). The response is then provided to a client device in 450 and process 400 is terminated in an end step 490.

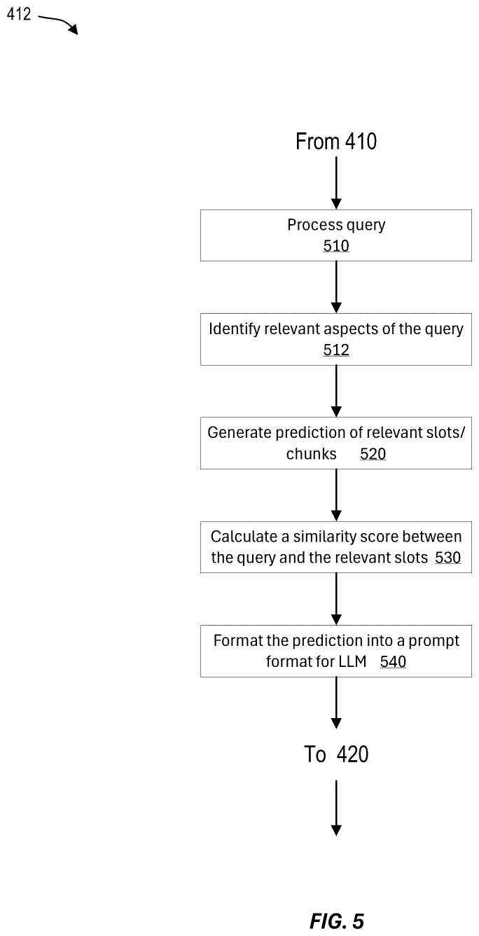

Further details of 412 to generate the input for the LLM is shown in FIG. 5. As shown in FIG. 5, the received query from 410 (i.e., query 110 in FIG. 1) is processed in 510 to extract content information, such as discussed above with respect to query understanding stage 124. The relevant aspects of the query, such as patient identification information, chief complaint, date of the patient encounter, and others, are identified in 512.

The relevant aspects of the query may then be used to generate a prediction of the relevant slots and/or chunks in the unstructured data (e.g., unstructured data 152 in EHR 150 of FIG. 1). In the illustrated example, a similarity score between the query and the relevant slots and/or chunks is calculated in 530, which information is then used to format the query and the prediction into a prompt format for use by the LLM in 540. The process proceeds to step 420 in process 400 of FIG. 4.

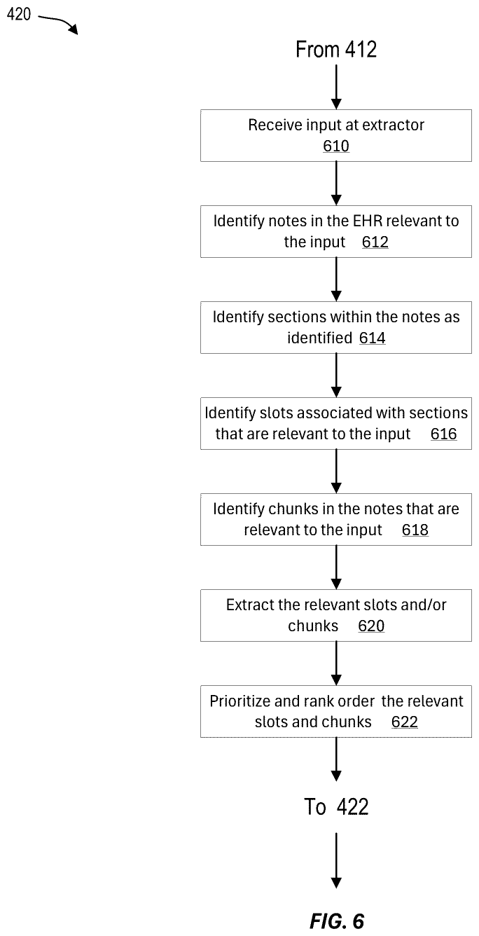

Further details of step 420 to identify the slots/chunks associated with the sections of content items based on the input are shown in FIG. 6. As shown in FIG. 6, at the extractor in 610, which then takes the information in the input to identify specific notes/content items in the EHR that may be relevant to the input in 612. Specific relevant sections within the content items are identified in 614, from which relevant slots are identified in 616. Specific relevant chunks within the notes/content items are identified in 618. As discussed above, a chunk may contain multiple slots, while a slot may contain multiple chunks, depending on the content provided in the input. The identified, relevant slots and/or chunks are extracted from the EHR in 620 then prioritized and rank ordered in 622 prior to being saved at the vector database in 422 of process 400 of FIG. 4 (e.g., in database 140).

It is recognized herein that such a multi-layer document chunking approach that consider generally standardized (although not specifically enriched with embeddings as such) sections to generate a vector database for optimization of RAG performance for large amounts of unstructured records is currently not available. The presently described techniques solve long standing problems recognized, for example, by clinicians who work with large amounts of unstructured data (e.g., patient records) within an EHR.

In particular, it is recognized herein that prediction of potentially relevant slots/chunks associated with document sections, population of a database with the relevant slots/chunks so identified, then selection of a subset of the slots/chunks as a part of generating the query result by the LLM is beyond a typical RAG implementation. Further, this two-stage generative process of selecting the potentially relevant slots/chunks (i.e., culling the range of information searched based on a processing of the query to extract context information), then using the LLM, rather than directly feeding the query into the LLM, is an approach that has not previously been available.

### Examples of Agentic Artificial Intelligence Architectures

The developed approach described herein addresses these challenges and others by providing techniques for assisting healthcare providers with necessary yet time-consuming and often tedious tasks. Techniques are disclosed herein for improving the efficiency of and reducing the computing resources required to perform various healthcare services in a clinical environment. In certain embodiments, techniques are disclosed for equipping a healthcare provider end user with a clinical software application that can be installed on and utilized from one or both of a mobile computing device and a desktop computing device to facilitate performance of the various tasks typically rendered by a healthcare provider as part of providing healthcare services to patients.

FIG. 7 shows a simplified diagram of a computing environment 700 incorporating agent-driven services. In examples, the agent-driven services may include one or more artificial intelligence resources acting as “agents,” each performing a defined set of tasks. For instance, one of the agents may implement the tailored RAG architecture for performing an improved search over unstructured data, as disclosed herein. In an embodiment, computing environment 700 includes one or more client devices 710 (hereinafter “client devices 710”), one or more communication channels 712 (hereinafter “communication channels 712”), a cloud services provider platform 714 (hereinafter “platform 714”) including agent-driven services 720 and connected with one or more databases 722 (hereinafter “databases 722”) and one or more large language models 724 (hereinafter “LLMs 724”).

As shown in FIG. 7, agent-driven services 720 may include, for example, one or more artificial intelligence agents (hereinafter “AI agent 726”). In an embodiment, agent driven services 720 includes a plurality of AI agents, shown as AI Agent 1 (726-1), AI Agent 2 (726-2), and so on, as indicated by ellipsis. Each AI agent may be trained to specialize in a particular task, such as the tailored RAG system implementation discussed above.

Platform 714 receives user query 705 from one of client devices 710 via communication channels 712, and user query 705 is passed to a planner 730. In embodiments, planner 730 may perform some or all of the functions of query understanding 124 described above. Further, planner 730 determines the appropriate course of action (e.g., selection of the appropriate AI agent with agent-driven services 720, timing and/or prioritization of tasks to be performed in response to the user query, etc.), then the action so determined is sent to executor 732.

In embodiments, at executor 732, a new execution plan may be generated or an existing plan selected out of a library of execution plans (not shown). An execution plan may include, for example, information regarding the course of action, timing, prioritization, etc. The execution plan is then implemented at one or more of the AI agents within agent-driven services 720. The one or more of the AI agents execute the appropriate tasks, based on the information accessible at databases 722 and LLMs 724, to send the resulting output to a response generator 740. Response generator 740 generates then transmits a response to the client device that originated the user query 705.

FIG. 8 shows a simplified diagram depicting an alternative computing environment 800 incorporating an agent-driven digital assistant system configured to generate knowledge-grounded response data according to certain embodiments. As shown in FIG. 8, the computing environment 800 includes one or more client devices 810 (hereinafter “client devices 810”), one or more communication channels 812 (hereinafter “communication channels 812”), a cloud service provider platform 814 (hereinafter “platform 814”), one or more databases 822 (hereinafter “databases 822”), and one or more LLMs 824 (hereinafter “LLMs 824”). The platform 814, which can be included as part of a cloud infrastructure of a cloud service provider (e.g., Oracle Cloud Infrastructure or OCI) as mentioned above, can be configured to communicate with, send data and information to, and receive data and information from the client devices 810 via the communication channels 812. Additionally, the platform 814 can be configured to access and/or call the databases 822 and the LLMs 824 to obtain and/or receive data and information from the databases 822 and the LLMs 824. Data and information received from the client devices 810, the databases 822, and the LLMs 824 can be used by the platform 814 to execute tasks and perform services such as automatically generating one or more portions of knowledge-grounded response data. While FIG. 8 shows the databases 822 and the LLMs 824 as being separate from the platform 814, this is not intended to be limiting, and one or more of the databases 822 and/or one or more of the LLMs 824 can be included as part of the platform 814 and/or the cloud infrastructure in which the platform 814 is included. While FIG. 8 describes the computing environment 800 as including the LLMs 824, other types of ML models can be included in the computing environment 800, such as an ML model configured for analyzing audio data and/or generating text based on audio data or an ML model configured to generate an execution plan for a group of multiple agent-driven services (or sub-services) included in the platform 814.

Each client device included in the client devices 810 can be any kind of electronic device that is capable of: executing applications; presenting information textually, graphically, and audibly such as via a display and a speaker; collecting information via one or more sensing elements such as image sensors, microphones, tactile sensors, touchscreen displays, and the like; connecting to a communication channel such as the communication channels 812 or a network such as a wireless network, wired network, a public network, a private network, and the like, to send and receive data and information; and/or storing data and information locally in one or more storage mediums of the electronic device and/or in one or more locations that are remote from the electronic device such as a cloud-based storage system, the platform 814, and/or the databases 822. Examples of electronic devices include, and are not limited to, mobile phones, desktop computers, portable computing devices, computers, workstations, laptop computers, tablet computers, and the like.

In some implementations, an application can be installed on, executing on, and/or accessed by a client device included in the client devices 810. The application and/or a user interface of the application can be utilized and/or interacted with (e.g., by an end user) to access, utilize, and/or interact with one or more services provided by the platform 814. The client devices 810 can be configured to receive multiple forms of input such as touch, text, voice, and the like, and the application can be configured to transform that input into one or more messages which can be transmitted or streamed to the platform 814 using one or more communication channels of the communication channels 812. Additionally, the client device can be configured to receive messages, data, and information from the platform 814 using one or more communication channels of the communication channels 812 and the application can be configured to present and/or render the received messages, data, and information in one or more user interfaces of the application. In some cases, the platform 814 receives one or more user queries, such as a user query 805, from the client devices 810. In some cases, the platform 814 provides one or more knowledge-grounded responses, such as knowledge-grounded response data 890, to the client devices 810.

Each communication channel included in the communication channels 812 can be any kind of communication channel that is capable of facilitating communication and the transfer of data and/or information between one or more entities such as the client devices 810, the platform 814, the databases 822, and the LLMs 824 (or other ML models). Examples of communication channels include, and are not limited to, public networks, private networks, the Internet, wireless networks, wired networks, fiber optic networks, local area networks, wide area networks, and the like. The communication channels 812 can be configured to facilitate data and/or information streaming between and among the one or more entities. In some implementations, data and/or information can be streamed using one or more messages and according to one or more protocols. Each of the one or more messages can be a variable length message and each communication channel included in the communication channels 812 can include a stream orchestration layer that can receive the variable length message in accordance with a predefined interface, such as an interface defined using an interface description language like AsyncAPI. Each of the variable length messages can include context information that can be used to determine the route or routes for the variable length message as well as a text or binary payload of arbitrary length. Each of the routes can be configured using a polyglot stream orchestration language that is agnostic to the details of the underlying implementation of the routing tasks and destinations.

Each database included in the databases 822 can be any kind of database that is capable of storing data and/or information and managing data and/or information. Data and/or information stored by each database can include data and/or information generated by, provided by, and/or otherwise obtained by the platform 814. Additionally, or alternatively, data and/or information stored and/or managed by each database can include data and/or information generated by, provided by, and/or otherwise obtained by other sources such as the client devices 810 and/or LLMs 824 (or other ML models). One or more databases that are included in the databases 822 can be part of a platform for storing and managing healthcare information such as electronic health records for patients, electronic records of healthcare providers, and the like, and can store and manage electronic health records for patients of healthcare providers. An example platform is the Oracle Health Millenium Platform. Additionally, one or more databases included in the databases 822 can be provided by, managed by, and/or otherwise included as part of a cloud infrastructure of a cloud service provider (e.g., Oracle Cloud Infrastructure or OCI). Data and/or information stored and/or managed by the databases 822 can be accessed using one or more application programming interfaces (APIs) of the databases 822.

Each LLM included in the LLMs 824 can be any kind of LLM that is capable of obtaining or generating or retrieving one or more results in response to one or more inputs such as one or more machine-learning prompts (hereinafter, “ML prompts” or “prompts”). ML prompts for obtaining or generating or retrieving results from the LLMs 824 can obtained from or generated by or retrieved from or accessed from the client devices 810, the databases 822, the platform 814, and/or one or more other sources such as the Internet. Each ML prompt can be configured to cause the LLMs 824 to perform one or more tasks, which causes one or more results to be provided or generated and the like. ML prompts for the LLMs 824 can be pre-generated (e.g., before they are needed for a particular task) and/or generated in real-time (e.g., without a delay noticeable to a human user). In some implementations, prompts for the LLMs 824 can be engineered to achieve a desired result or results manually and/or by one or more ML models. In some implementations, ML prompts for the LLMs 824 can be engineered on demand (i.e., in real-time and/or as needed) and/or at particular intervals (e.g., once per day, upon log in by authenticated user into the platform 814). Each ML prompt of the one or more ML prompts can include a request, such as a query, for a task to be performed by the LLMs 824. In some cases, an ML prompt can include additional information, such as data generated by one or more services (e.g., agent-driven services) included in the platform 814. The additional information can include information such as one or more ML prompt templates, structured data that is configured to be interpreted (e.g., semantically interpreted) by a computing system component (e.g., an ML model, an agent-driven service, etc.), unstructured data that is configured to be interpreted (e.g., semantically interpreted) by a human, responses from one or more ML models, output data generated by one or more agent-driven services, and/or other information suitable to include in an ML prompt. LLMs included in the LLMs 824 can be pre-trained, fine-tuned, open source, off-the-shelf, licensed, subscribed to, and the like. Additionally, LLMs included in the LLMs 824 can include or have any size context window (e.g., can accept any number of tokens) and can be capable of interpreting complex instructions. One or more LLMs included in the LLMs 824 can be provided by, managed by, and/or otherwise included as part of the platform 814 and/or a cloud infrastructure of a cloud service provider (e.g., Oracle Cloud Infrastructure or OCI) that supports the platform 814. One or more LLMs included in the LLMs 824 can be accessed using one or more APIs of the LLMs 824 and/or a platform hosting or supporting or providing the LLMs 824. In some implementations, one or more additional ML models included in the environment 800 may have one or more characteristics that are similar to characteristics described in regard to the LLMs 824.

The platform 814 can be configured to include various capabilities and provide various services to subscribers (e.g., end users) of the various services. In some implementations, such as in the case of an end user or subscriber being a healthcare provider, the healthcare provider can utilize the various services to facilitate the observation, care, treatment, management, and so on of their patient populations. For example, a healthcare provider can utilize the functionality provided by the various services provided by the platform 814 to examine and/or treat and/or facilitate the examination and/or treatment of a patient; view, edit, and/or manage a patient's electronic health record; perform administrative tasks such as placing medical orders, scheduling appointments, managing patient populations, providing customer service to facilitate operation of a healthcare environment in which the healthcare provider practices, and so on.

In some implementations, the services provided by the platform 814 can include, and are not limited to, a response engine 816 and a knowledge engine 818. In some implementations, one or more services provided by the platform 814, such as the response engine 816 and/or the knowledge engine 818, can be configured to operate as agent-driven services, such as agent-driven services 820. In some implementations, an execution plan guides activities of one or more of the agent-driven services 820 provided by the platform 814. For example, the platform 814 can include, such as included in or in addition to the LLMs 824, a generative AI model (or another suitable ML model included in the environment 800) that is configured to generate (or modify) an execution plan. In this example, the execution plan can describe actions associated with one or more of the agent-driven services 820 provided by the platform 814. Based on the execution plan generated by the example generative AI model, one or more of the agent-driven services 820 can be configured to operate and/or interact with one or more additional ones of the agent-driven services 820. In some implementations, an output of the platform 814 is described by the execution plan. For example, the example generative AI model could be configured to generate an execution plan based on request data associated with the platform 814 (such as request data included in at least one query received by the platform 814 from one or more of the client devices 810). In some cases, the platform 814 and/or the example generative AI model can determine that the request data is associated with at least one agent-driven service of the services 820 provided by the platform 814, such as request data associated with the response engine 816 and/or the knowledge engine 818. In this example, the example generative AI model can generate an execution plan that describes one or more agent tasks for the at least one agent-driven service provided by the platform 814, such as agent tasks for generating a response to a user query and/or generating knowledge-grounded response data. For example, the response engine 816, the knowledge engine 818, and/or additional services in the agent-driven services 820 generates at least one response data object, such as the knowledge-grounded response data 890, based on a combination of multiple data outputs from the response engine 816 and/or the knowledge engine 818. In this example, the knowledge engine 818 generates the knowledge-grounded response data 890 by combining multiple data outputs from the response engine 816 and/or the knowledge engine 818 in a combination that is described by the execution plan.

In some implementations, the generated execution plan can omit instructions for implementing an agent task and include data outlining an agent task (e.g., data outlining one or more data sources, inputs, or requested outputs). In some implementations, one or more service of the agent-driven services 820 can generate its own instructions for implementing an agent task based on the data outlined in the execution plan. For example, an agent-driven service included in (or otherwise associated with) the response engine 816 can construct one or more ML prompts for generating response data, such as by using data outlined in the example execution plan to identify a prompt template (e.g., from a library of templates), a data source (e.g., a data repository storing information related to the user query 805), and one or more of the LLMs 824 (e.g., configured to generate text data summarizing medical information related to the user query 805). As another example, an agent-driven service included in (or otherwise associated with) the knowledge engine 818 can construct one or more ML prompts for generating and/or annotating the knowledge-grounded response data 890, such as by using data outlined in the example execution plan to identify a prompt template, a data source (e.g., data summarized by the response engine 816 and/or the data repository storing information related to the user query 805), and one or more of the LLMs 824 (e.g., configured to determine at least one response annotation using the data summarized by the response engine 816). In some cases, based on the data outlined in the execution plan, the response engine 816 and/or the knowledge engine 818 can be configured to generate respective instructions by which the response engine 816 and/or 818 can operate and/or interact with one or more additional services provided by the platform 814 (such as, and not limited to, additional services of the agent-driven services 820). Examples of skill-driven and LLM-based and agent-driven digital assistants are described in U.S. patent application Ser. No. 17/648,376, filed on Jan. 19, 2022, and U.S. patent application Ser. No. 18/624,472, filed on Apr. 2, 2024, each of which are incorporated by reference as if fully set forth herein.

In the platform 814, one or more of the agent-driven services 820 are configured, such as based on one or more generated execution plans, to create and/or annotate one or more portions of the knowledge-grounded response data 890. In the computing environment 800, the agent-driven services 820 can create the knowledge-grounded response data 890 in response to receiving one or more queries, such as a user query 805. To create the knowledge-grounded response data 890, the agent-driven services 820 perform, via the platform 814, one or more of acquiring LLMs, execution plan creation and/or implementation, asset identification (such as identification of one or more model-selected assets 850), and providing the knowledge-grounded response data 890 to one or more additional computing systems, such as to the client devices 810. For example, the platform 814 may receive the user query 805 from a particular one of the client devices 810. In addition, the platform 814 may generate at least one execution plan based on the user query 805. In some cases, one or more of the response engine 816, the knowledge engine 818, or one or more additional services of the agent-driven services 820 may identify at least one of the LLMs 824 based on the execution plan (or respective portions of the execution plan). In addition, one or more of the response engine 816, the knowledge engine 818, or the one or more additional services of the agent-driven services 820 may identify, such as from the databases 822, at least one asset based on the execution plan (or respective portions of the execution plan). Based on the identified one(s) of the LLMs 824 and/or the identified asset(s), one or more of the response engine 816, the knowledge engine 818, or the one or more additional services of the agent-driven services 820 may generate and/or modify the knowledge-grounded response data 890. In some cases, the knowledge-grounded response data 890 includes a combination of response data and attention cue data, such as response data that responds to a question (or other query type) included in the user query 805 and attention cue data that draws a user's attention to at least a portion of the response data. Examples of response data can include text data, numeric data, image data (e.g., a radiology image), tabulated data (e.g., arranged in a table or other suitable format), or other types of response data suitable for responding to a user query. Examples of attention cue data can include highlighting data (e.g., color text, color background, color-vision deficiency patterns, etc.), font data (e.g., font size, italics, bold, underlining, typeface, etc.), audio data (e.g., automatic speech generation, audible alert data, etc.), haptic data (e.g., vibration, etc.), or other suitable types of attention cue data suitable for drawing user attention to at least a portion of response data.

In some implementations, the response engine 816 can be configured to automatically generate some or all response data that is included in the knowledge-grounded response data 890. For example, by utilizing an execution plan that is generated based on the user query 805, the response engine 816 may identify a first LLM from the LLMs 824 and one or more assets from the databases 822, such as one or more of an asset 850A, an asset 850B, through an asset 850N that are included in the model-selected assets 850. In addition, the response engine 816 may generate one or more ML prompts based on the one or more assets and provide the one or more ML prompts to the first LLM. Based on information received from the first LLM (e.g., in response to the one or more ML prompts), the response engine 816 may generate response data that responds to a question included in the user query 805. For example, if the user query 805 includes a question “How has Ms. Henderson's new blood pressure medication been working?” the response engine 816 may identify, such as from an electronic health record (hereinafter, “EHR”) associated with the patient Ms. Henderson, a group of blood pressure measurements from a time period associated with a blood pressure medication currently prescribed to the patient. The response engine 816 may select the group of blood pressure measurements as information included in the asset 850A. In some cases, the response engine 816 may identify one or more additional assets from the databases 822 and include the additional assets in the model-selected assets 850, such as including in the asset 850B information describing the currently prescribed blood pressure medication or including in the asset 850N information describing additional medical factors for the patient (e.g., an additional diagnosis, a preferred exercise frequency for the patient, etc.) Continuing with this example, the response engine 816 may determine that the first LLM is fine-tuned to summarize information. In addition, the response engine 816 may generate a first ML prompt that includes one or more of the identified assets (e.g., assets 850A through 850N) and provide the first ML prompt to the first LLM. Based on data received from the first LLM, e.g., data summarizing the identified assets included in the first ML prompt, the response engine 816 may generate response data that includes a combination of text and tabulated numeric data, such as a table of blood pressure measurements and a text description of a trend in the blood pressure measurements since the patient Ms. Henderson began taking the currently prescribed blood pressure medication. In addition, the response engine 816 may generate or modify the knowledge-grounded response data 890 to include the response data including the combination of text and tabulated numeric data.

In some implementations, the knowledge engine 818 can be configured to automatically generate some or all attention cue data that is included in the knowledge-grounded response data 890. For example, by utilizing the execution plan that is generated based on the user query 805, the knowledge engine 818 may identify a second LLM from the LLMs 824. In addition, the knowledge engine 818 may identify one or more assets, such as one or more the response data generated by the response engine 816 and/or one or more of the assets 850A through 850N. In some cases, the knowledge engine 818 may generate one or more ML prompts based on the one or more assets and provide the one or more ML prompts to the second LLM. Based on information received from the second LLM (e.g., in response to the one or more ML prompts), the knowledge engine 818 may generate attention cue data that draws user attention to at least a portion of the response data generated by the response engine 816. Continuing with the example question “How has Ms. Henderson's new blood pressure medication been working?” the knowledge engine 818 may determine that the second LLM is fine-tuned to identify high-relevance data in one or more assets. In addition, the knowledge engine 818 may generate a second ML prompt that includes one or more of the identified assets (e.g., the response data generated by the response engine 816 and the assets 850A through 850N) and provide the second ML prompt to the second LLM. Based on data received from the second LLM, e.g., data identifying high-relevance data included in the second ML prompt, the knowledge engine 818 may generate attention cue data that draws attention to the high-relevance data, such as color highlighting that draws attention to a trend in the blood pressure measurements and a bold font style that draws attention to information describing a possible interaction of the currently prescribed blood pressure medication with an additional medication frequently prescribed for an additional diagnosis of the patient. In addition, the knowledge engine 818 may generate or modify the knowledge-grounded response data 890 to include the attention cue data, e.g., modifying the knowledge-grounded response data 890 to apply the color highlighting to at least a portion of the tabulated numeric data and the bold font style to at least a portion of the text data. In some cases, the knowledge engine 818 may modify the knowledge-grounded response data 890 to include additional response data, such as interactive reference data (e.g., a uniform resource locator (URL) address) that provides one or more references describing a source of information included in the knowledge-grounded response data 890. Continuing with the above example, the knowledge engine 818 may generate first interactive reference data that provides a first reference to one or more EHRs including blood pressure measurements for the patient. In addition, the knowledge engine 818 may generate second interactive reference data that provides a second reference to medication information (e.g., a medication reference database) describing possible interactions of the currently prescribed blood pressure medication.

In some implementations, the platform 814 (or a component thereof) may provide the knowledge-grounded response data 890 to one or more additional computing systems. For example, the platform 814 may identify a particular client device of the client devices 810 from which the user query 805 was received. In addition, the platform 814 may provide the knowledge-grounded response data 890 to the particular client device. In some cases, the particular client device is configured to perform one or more operations based on the knowledge-grounded response data 890, such as operations related to displaying the combination of the response data and the attention cue data included in the knowledge-grounded response data 890. For example, the particular client device may be configured to display the response data as annotated by the attention cue data, e.g., the table of blood pressure measurements and the text description as annotated by the color highlighting, the bold font style, and the interactive reference data. In addition, the particular client device may be configured to receive additional input data based on the knowledge-grounded response data 890, such as a user input indicating a selection of at least a portion of the knowledge-grounded response data 890. For example, responsive to receiving a user selection input of the first interactive reference data, the particular client device may be configured to send, to the platform 814, a request to receive at least a portion of the first reference, such as the one or more EHRs (or a portion thereof) including blood pressure measurements for the patient. In addition, responsive to receiving an additional user selection input of the second interactive reference data, the particular client device may be configured to send, to the platform 814, an additional request to receive at least a portion of the second reference, such as the medication information (or a portion thereof) describing possible interactions of the currently prescribed blood pressure medication. In some cases, the data architecture and/or configuration of the platform 814, such as the combination of the response engine 816 and the knowledge engine 818 and/or combination of some or all described features thereof, can improve user comprehension of information provided in response to user queries, such as the user query 805. For example, the combination of the response data with the attention cue data, such as included in the knowledge-grounded response data 890, can improve comprehension or reduce reading time by a user, such as by drawing the user's attention to portions of the response data that are annotated by the attention cue data. In addition, the combination of the response data with the interactive reference data that is included in the attention cue data, such as included in the knowledge-grounded response data 890, can improve user trust in the response data by facilitating fast identification of potentially inaccurate data (e.g., hallucinations) generated by one or more of the LLMs 824, such as by providing fast access to reference information via the interactive reference data.

In FIG. 8, the response engine 816 and the knowledge engine 818 are described as utilizing a particular execution plan (e.g., respective portions of a same execution plan), and other implementations are possible. For example, a cloud service provider platform may generate a respective execution plan for each particular agent-driven service that is included in (or otherwise utilized by) the example cloud service provider platform. In FIG. 8, the response engine 816 and the knowledge engine 818 are described as respectively identifying the first LLM and the second LLM from the LLMs 824, and other implementations are possible. For example, in various instances, the response engine 816 and the knowledge engine 818 (or others of the agent-driven services 820) may identify from the LLMs 824 at least one same LLM, at least one different LLM, and/or a combination of different and same LLMs.

In some instances, the agent-driven services 820 can be utilized to access pre-trained and/or fine-tuned ML models, such as one or more of the LLMs 824. The pre-trained ML models serve as foundational elements, possessing extensive language understanding derived from vast datasets. This capability enables the models to generate coherent responses across various topics, facilitating transfer learning. Pre-trained models offer cost-effectiveness and flexibility, which allows for scalable improvements and continuous pre-training with new data, often establishing benchmarks in natural language processing tasks. Conversely, fine-tuned models are specifically trained for tasks or industries (e.g., plan creation utilizing the LLM's in-context learning capability, knowledge or information retrieval on behalf of an agent, response generation for human-like conversation, etc.), enhancing their performance on specific applications and enabling efficient learning from smaller, specialized datasets. Fine-tuning provides advantages such as task specialization, data efficiency, quicker training times, model customization, and resource efficiency. In some embodiments, fine-tuning may be particularly advantageous for niche applications and ongoing enhancement. In other instances, the agent-driven services 820 can be utilized to pre-train and/or fine-tune the LLMs. The agent-driven services 820, or any subset thereof, may be standalone or part of a machine-learning operationalization framework, inclusive of hardware components like processors (e.g., CPU, GPU, TPU, FPGA, or any combination), memory, and storage. This framework operates software or computer program instructions (e.g., TensorFlow, PyTorch, Keras, etc.) to execute arithmetic, logic, input/output commands for training, validating, and deploying machine-learning models in a production environment. In certain instances, the agent-driven services 820 implement the training, validating, and deploying of the models using a cloud platform such as Oracle Cloud Infrastructure (OCI). In some cases, leveraging a cloud platform can make machine-learning more accessible, flexible, and cost-effective, which can facilitate faster model development and deployment for developers.

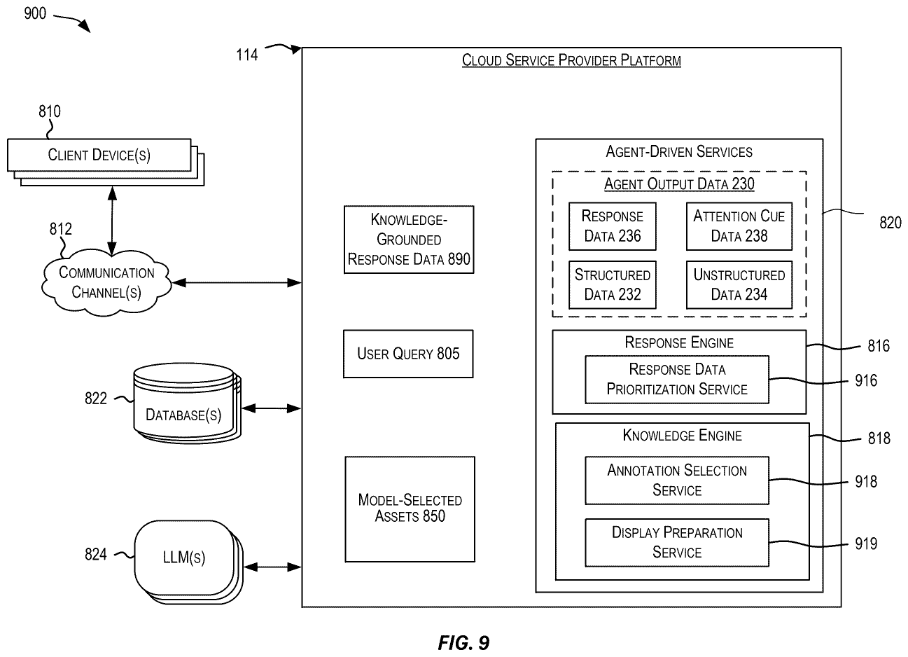

Although not shown, the platform 814 can include other capabilities and services such as authentication services, management services, task management services, notification services, and the like. The various capabilities and services of the platform 814 can be implemented utilizing one or more computing resources and/or servers of the platform 814 and provided by the platform 814 by way of subscriptions. Additionally, or alternatively, while FIG. 8 shows the services of the platform 814 as being separate services, one or more of the services can be combined with other services and/or be considered to be a sub-service of another service. In some implementations, a particular service in the agent-driven services 820 may utilize an output from another service in the agent-driven services 820, such as to facilitate quick completion of one or more operations by the particular service. For example, as shown in FIG. 9, in a computing environment 900, one or more of the response engine 816 or the knowledge engine 818 can include at least one sub-service. In addition, one or more of the response engine 816 or the knowledge engine 818 can access one or more outputs from one or more services of the agent-driven services 820, such as agent output data 930. In some cases, the availability of the agent output data 930 to multiple services in the agent-driven services 820, such as at least the response engine 816 and the knowledge engine 818, can improve response time by the multiple services in the agent-driven services 820. For example, the response engine 816 and the knowledge engine 818 may provide one or more outputs with decreased response time and decreased use of computing resources (e.g., processing resources, memory resources, ML model resources, etc.) by using some or all of the agent output data 930 as input data.

In the computing environment 900, the agent output data 930 includes, at least, structured data 932, unstructured data 934, response data 936, and attention cue data 938. FIG. 9 depicts the structured data 932, the unstructured data 934, the response data 936, and the attention cue data 938 as being included in the agent output data 930, and other implementations are possible, such as one or more of the data 932, 934, 936, or 938 being included in the model-selected assets 850. In some cases, the response data 936 is an output from the response engine 816 (or a sub-service thereof) and the attention cue data 938 is an output from the knowledge engine 818 (or a sub-service thereof). In some cases, one or more of the structured data 932 and the unstructured data 934 is an output from one or more additional services of the agent-driven services 820, such as additional services configured for generating text data based on audio data (e.g., transcription of spoken conversation between a patient and a healthcare provider), identifying EHRs for a particular patient, or other tasks suitable to be performed by the agent-driven services 820. In FIG. 9, the structured data 932 can include data that is structured to be interpreted by a computing device, such as database records, JavaScript data objects, or other data objects (e.g., included in one or more EHRs) that are intended for computer interpretation (e.g., not intended for human interpretation). In FIG. 9, the unstructured data 934 can include data that lacks a structure interpreted by a computing device, such as patient appointment notes (e.g., Subjective, Objective, Assessment, and Plan notes, “SOAP notes”), medical reference materials (e.g., medication documentation, surgical procedure guidelines, etc.), or other types of information that are intended for human interpretation.

In the computing environment 900, the response engine 816 can include at least one sub-service, such as a response data prioritization service 916. In some cases, the response data prioritization service 916 may access at least one of the agent output data 930, such as one or more of the structured data 932 or the unstructured data 934. In addition, the response data prioritization service 916 may evaluate portions of the structured data 932 or the unstructured data 934 for potential inclusion in response data, such as potential inclusion in the response data 936 and/or in the knowledge-grounded response data 890. For example, the response data prioritization service 916 may evaluate a data portion to determine whether the data is relevant to a question or other information in the user query 805. In some cases, the response data prioritization service 916 may send one or more portions of the structured data 932 or the unstructured data 934 to at least one LLM of the LLMs 824, such as in an ML prompt. In some implementations, the ML prompt also includes data corresponding to the user query 805, such as a question included in the user query 805 or additional data (e.g., determined by a service in the agent-driven services 820), such as additional data corresponding to the user query 805.

In the computing environment 900, the response data prioritization service 916 may receive, from the at least one LLM, multiple data portions that are extracted from one or more of the structured data 932 or the unstructured data 934. In addition, the response data prioritization service 916 may determine a relevance status for each of the multiple data portions, such as by comparing each data portion to one or more relevance threshold values. In some cases, the response data prioritization service 916 may determine, such as by determining a similarity (e.g., semantic similarity) between each data portion and query data (e.g., information associated with the user query 805), whether each data portion exceeds (or fulfill another relationship with) the one or more relevance threshold values. For example, based on a comparison to a first relevance threshold value, the response data prioritization service 916 may identify various data portions as high-relevance data, such as high-relevance data that directly answers one or more questions included in the user query 805. In some cases, the response data prioritization service 916 may select one or more of the LLMs 824 to modify some of the high-relevance data before inclusion in the response data 936 and/or the knowledge-grounded response data 890. For example, the response data prioritization service 916 may provide a first portion of the high-relevance data to a first LLM (e.g., from the LLMs 824) that is fine-tuned to summarize received information in text summary data. Examples of text summary data can include paragraphs, single sentences, or other human-readable text data that summarizes larger amounts of information. In addition, the response data prioritization service 916 may modify the response data 936 and/or the knowledge-grounded response data 890 to include the text summary data which summarizes the high-relevance data. As another example, the response data prioritization service 916 may provide a second portion of the high-relevance data to a second LLM (e.g., from the LLMs 824) that is fine-tuned to arrange received information as tabulated data, such as multiple items of information that are intended to be interpreted as a group. Examples of tabulated data can include tables, bulleted lists, numbered lists, or other organized arrangements of multiple items of information intended to be interpreted as a group. Examples of information that could be arranged as tabulated data can include a group of lab results, a group of comparison medications (e.g., generics, non-generics, etc.), a group of potential side effects of a surgical procedure, or other groups of information items. In addition, the response data prioritization service 916 may modify the response data 936 and/or the knowledge-grounded response data 890 to include the tabulated data in which the high-relevance data is arranged.

In some implementations, based on a comparison to a second relevance threshold value, the response data prioritization service 916 may identify one or more data portions (e.g., extracted from one or more of the structured data 932 or the unstructured data 934) as medium-relevance data. In some cases, the medium-relevance data can include supplemental data that does not directly answer one or more questions included in the user query 805 and which provides additional data about a topic identified in the one or more questions. Examples of supplemental data can include information about a diagnosed condition, information about a patient circumstance (e.g., a high-exercise lifestyle, a preference to avoid injected medications, etc.), or other types of information that are generally related to a question. In some cases, the response data prioritization service 916 may select one or more of the LLMs 824 to modify some of the medium-relevance data before inclusion in the response data 936 and/or the knowledge-grounded response data 890. For example, the response data prioritization service 916 may provide a portion of the medium-relevance data to the first LLM that is fine-tuned to summarize received information in text summary data. In addition, the response data prioritization service 916 may modify the response data 936 and/or the knowledge-grounded response data 890 to include additional text summary data which summarizes the medium-relevance data.

In the computing environment 900, the knowledge engine 818 can include at least one sub-service, such as one or more of an annotation selection service 918 or a display preparation service 919. In some cases, one or more of the annotation selection service 918 or the display preparation service 919 may access at least one of the agent output data 930, such as the response data 936 that is generated by the response engine 816. For example, the annotation selection service 918 may generate the attention cue data 938 that annotates some or all of the response data 936. In addition, the display preparation service 919 may generate computer-implemented instructions (e.g., markup language, executable code, etc.) that can be implemented via one or more computing devices to display the attention cue data 938, or a combination of the attention cue data 938 with the response data 936. Examples of computer-implemented instructions related to display of attention cue data can include hypertext markup language (HTML) instructions, extensible markup language (XML) instructions, or other suitable types of instructions for implementing data display (e.g., visual display, audio display, etc.) via one or more user interface devices.

In the computing environment 900, the annotation selection service 918 may provide one or more portions of the response data 936 to at least one LLM of the LLMs 824, such as in an ML prompt. In some implementations, the ML prompt also includes additional data (e.g., determined by a service in the agent-driven services 820), such as additional data corresponding to one or more of the user query 805, the structured data 932, or the unstructured data 934. For example, the at least one LLM may be fine-tuned to identify, in the response data 936, one or more portions of data that have a relatively high similarity to data included in one or more of the user query 805, the structured data 932, or the unstructured data 934, such as a portion of high-relevance text summary data in the response data 936 that has a high similarity to text data of a question included in the user query 805. In some cases, the annotation selection service 918 may receive, from the at least one LLM, data identifying at least one portion of the response data 936 for annotation. In addition, the annotation selection service 918 may determine one or more types of annotations to apply to the identified portion of the response data 936, such as annotations for highlighting, font styles, or other types of annotations that can be applied to response data. In some implementations, the annotation selection service 918 may determine at least one type of annotation that applies interactive reference data to the identified portion of the response data 936. For example, the annotation selection service 918 may determine one or more sources for the identified portion of the response data 936, such as a source document and/or source database associated with one or more of the structured data 932 or the unstructured data 934. Based on the determined one or more sources, the annotation selection service 918 may generate interactive reference data that indicates the source(s) for the identified portion of the response data 936. In some cases, the annotation selection service 918 may identify source address data associated with the source(s) for the identified portion of the response data 936. Examples of source address data can include a network address (e.g., a URL, a MAC address), computing component identification data (e.g., identification of a particular database, etc.), document identification data (e.g., identification of a particular document, identification of a section within a document, etc.), or other types of address data that can identify a location (or other identification type) for a source repository. In addition, the annotation selection service 918 may include the source address data in the generated interactive reference data. In some cases, the annotation selection service 918 may generate or modify the attention cue data 938 to include (or otherwise indicate) the types of annotations and/or portions of the response data 936 to which the annotations are applied.

In the computing environment 900, the display preparation service 919 may identify one or more associated portions of response data and attention cue data, such as portions of the response data 936 that are associated with portions of the attention cue data 938. In addition, the display preparation service 919 may generate one or more computer-implemented instructions that combine the associated portions of response data and attention cue data for presentation via one or more user interface devices. For example, the display preparation service 919 may determine an association between a first portion of the response data 936 that indicates text data, such as a sentence, and a first portion of the attention cue data 938 that indicates an annotation, such as a bold typeface. In addition, the display preparation service 919 may generate at least one computer-implemented instruction that combines the associated portions, such as an HTML instruction (or other suitable instruction type) that applies the bold typeface to the sentence. As another example, the display preparation service 919 may determine an additional association between a second portion of the response data 936 that indicates tabulated data, such as a set of blood pressure measurements, and a second portion of the attention cue data 938 that indicates an additional annotation including interactive reference data, such as a patient chart that is a source document for the set of blood pressure measurements. In addition, the display preparation service 919 may generate at least one additional computer-implemented instruction that combines the additional associated portions, such as an additional HTML instruction (or other suitable instruction type) that applies an interactive link to the tabulated set of blood pressure measurements, e.g., the interactive link is directed to the patient chart. In some cases, the display preparation service 919 may generate or modify the knowledge-grounded response data 890 to include (or otherwise indicate) the computer-implemented instruction that combines the associated portions of the response data 936 and the attention cue data 938.

The computing environments 800 and 900 depicted in FIGS. 8 and 9 are merely exemplary and are not intended to unduly limit the scope of claimed embodiments. One of ordinary skill in the art would recognize many possible variations, alternatives, and modifications. For example, in some implementations, the computing environments 800 and 900 can be implemented using more or fewer services than those shown in FIGS. 8 and 9, may combine two or more services, or may have a different configuration or arrangement of services.

### Examples of Cloud Infrastructure Architectures

The term cloud service is generally used to refer to a service that is made available by a cloud service provider (CSP) to users (e.g., cloud service customers) on demand (e.g., via a subscription model) using systems and infrastructure (cloud infrastructure) provided by the CSP. Typically, the servers and systems that make up the CSP's infrastructure are separate from the user's own on-premise servers and systems. Users can thus avail themselves of cloud services provided by the CSP without having to purchase separate hardware and software resources for the services. Cloud services are designed to provide a subscribing user easy, scalable access to applications and computing resources without the user having to invest in procuring the infrastructure that is used for providing the services.

There are several cloud service providers that offer various types of cloud services. As discussed herein, there are various types or models of cloud services including IaaS, software as a service (SaaS), platform as a service (PaaS), and others. A user can subscribe to one or more cloud services provided by a CSP. The user can be any entity such as an individual, an organization, an enterprise, and the like. When a user subscribes to or registers for a service provided by a CSP, a tenancy or an account is created for that user. The user can then, via this account, access the subscribed-to one or more cloud resources associated with the account.

As noted above, infrastructure as a service (IaaS) is one particular type of cloud computing. IaaS can be configured to provide virtualized computing resources over a public network (e.g., the Internet). In an IaaS model, a cloud computing provider can host the infrastructure components (e.g., servers, storage devices, network nodes (e.g., hardware), deployment software, platform virtualization (e.g., a hypervisor layer), or the like). In some cases, an IaaS provider may also supply a variety of services to accompany those infrastructure components (example services include billing software, monitoring software, logging software, load balancing software, clustering software, etc.). Thus, as these services may be policy-driven, IaaS users may be able to implement policies to drive load balancing to maintain application availability and performance.

In some instances, IaaS customers may access resources and services through a wide area network (WAN), such as the Internet, and can use the cloud provider's services to install the remaining elements of an application stack. For example, the user can log in to the IaaS platform to create virtual machines (VMs), install operating systems (OSs) on each VM, deploy middleware such as databases, create storage buckets for workloads and backups, and even install enterprise software into that VM. Customers can then use the provider's services to perform various functions, including balancing network traffic, troubleshooting application issues, monitoring performance, managing disaster recovery, etc.

In most cases, a cloud computing model will require the participation of a cloud provider. The cloud provider may, and need not be, a third-party service that specializes in providing (e.g., offering, renting, selling) IaaS. An entity might also opt to deploy a private cloud, becoming its own provider of infrastructure services.

In some examples, IaaS deployment is the process of putting a new application, or a new version of an application, onto a prepared application server or the like. It may also include the process of preparing the server (e.g., installing libraries, daemons, etc.). This is often managed by the cloud provider, below the hypervisor layer (e.g., the servers, storage, network hardware, and virtualization). Thus, the customer may be responsible for handling (OS), middleware, and/or application deployment (e.g., on self-service virtual machines (e.g., that can be spun up on demand)) or the like.

In some examples, IaaS provisioning may refer to acquiring computers or virtual hosts for use, and even installing needed libraries or services on them. In most cases, deployment does not include provisioning, and the provisioning may need to be performed first.

In some cases, there are two different challenges for IaaS provisioning. First, there is the initial challenge of provisioning the initial set of infrastructure before anything is running. Second, there is the challenge of evolving the existing infrastructure (e.g., adding new services, changing services, removing services, etc.) once everything has been provisioned. In some cases, these two challenges may be addressed by enabling the configuration of the infrastructure to be defined declaratively. In other words, the infrastructure (e.g., what components are needed and how they interact) can be defined by one or more configuration files. Thus, the overall topology of the infrastructure (e.g., what resources depend on which, and how they each work together) can be described declaratively. In some instances, once the topology is defined, a workflow can be generated that creates and/or manages the different components described in the configuration files.

In some examples, an infrastructure may have many interconnected elements. For example, there may be one or more virtual private clouds (VPCs) (e.g., a potentially on-demand pool of configurable and/or shared computing resources), also known as a core network. In some examples, there may also be one or more inbound/outbound traffic group rules provisioned to define how the inbound and/or outbound traffic of the network will be set up and one or more virtual machines (VMs). Other infrastructure elements may also be provisioned, such as a load balancer, a database, or the like. As more and more infrastructure elements are desired and/or added, the infrastructure may incrementally evolve.

In some instances, continuous deployment techniques may be employed to enable deployment of infrastructure code across various virtual computing environments. Additionally, the described techniques can enable infrastructure management within these environments. In some examples, service teams can write code that is desired to be deployed to one or more, and often many, different production environments (e.g., across various different geographic locations, sometimes spanning the entire world). However, in some examples, the infrastructure on which the code will be deployed must first be set up. In some instances, the provisioning can be done manually, a provisioning tool may be utilized to provision the resources, and/or deployment tools may be utilized to deploy the code once the infrastructure is provisioned.

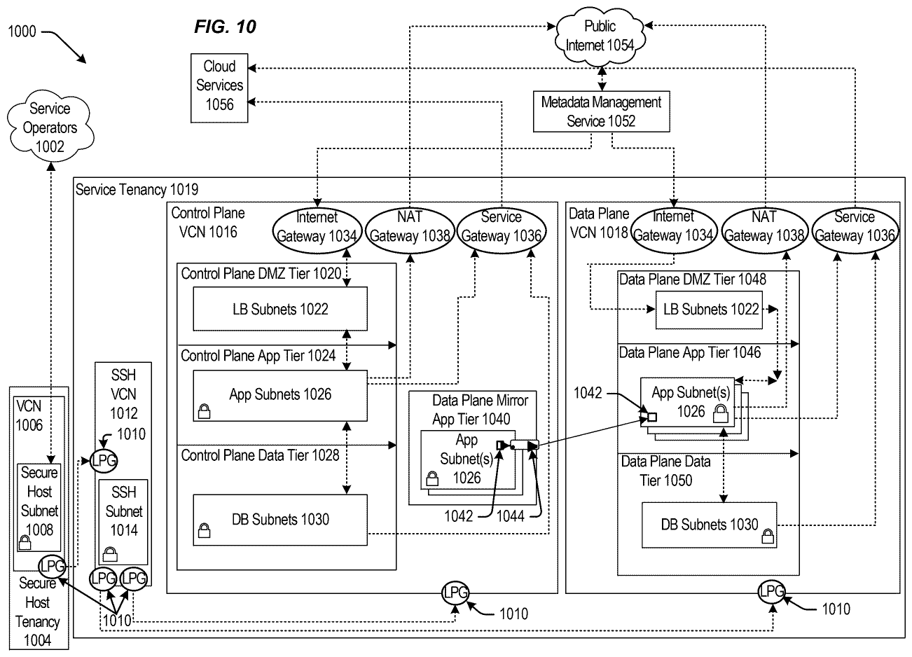

FIG. 10 is a block diagram 1000 illustrating an example pattern of an IaaS architecture, according to at least one embodiment. Service operators 1002 can be communicatively coupled to a secure host tenancy 1004 that can include a virtual cloud network (VCN) 1006 and a secure host subnet 1008. In some examples, the service operators 1002 may be using one or more client computing devices, which may be portable handheld devices (e.g., an iPhone®, cellular telephone, an iPad®, computing tablet, a personal digital assistant (PDA)) or wearable devices (e.g., a Google Glass® head mounted display), running software such as Microsoft Windows Mobile®, and/or a variety of mobile operating systems such as iOS, Windows Phone, Android, BlackBerry 9, Palm OS, and the like, and being Internet, e-mail, short message service (SMS), Blackberry®, or other communication protocol enabled. Alternatively, the client computing devices can be general purpose personal computers including, by way of example, personal computers and/or laptop computers running various versions of Microsoft Windows®, Apple Macintosh®, and/or Linux operating systems. The client computing devices can be workstation computers running any of a variety of commercially-available UNIX® or UNIX-like operating systems, including without limitation the variety of GNU/Linux operating systems, such as for example, Google Chrome OS. Alternatively, or in addition, client computing devices may be any other electronic device, such as a thin-client computer, an Internet-enabled gaming system (e.g., a Microsoft Xbox gaming console with or without a Kinect® gesture input device), and/or a personal messaging device, capable of communicating over a network that can access the VCN 1006 and/or the Internet.

The VCN 1006 can include a local peering gateway (LPG) 1010 that can be communicatively coupled to a secure shell (SSH) VCN 1012 via an LPG 1010 contained in the SSH VCN 1012. The SSH VCN 1012 can include an SSH subnet 1014, and the SSH VCN 1012 can be communicatively coupled to a control plane VCN 1016 via the LPG 1010 contained in the control plane VCN 1016. Also, the SSH VCN 1012 can be communicatively coupled to a data plane VCN 1018 via an LPG 1010. The control plane VCN 1016 and the data plane VCN 1018 can be contained in a service tenancy 1019 that can be owned and/or operated by the IaaS provider.

The control plane VCN 1016 can include a control plane demilitarized zone (DMZ) tier 1020 that acts as a perimeter network (e.g., portions of a corporate network between the corporate intranet and external networks). The DMZ-based servers may have restricted responsibilities and help keep breaches contained. Additionally, the DMZ tier 1020 can include one or more load balancer (LB) subnet(s) 1022, a control plane app tier 1024 that can include app subnet(s) 1026, a control plane data tier 1028 that can include database (DB) subnet(s) 1030 (e.g., frontend DB subnet(s) and/or backend DB subnet(s)). The LB subnet(s) 1022 contained in the control plane DMZ tier 1020 can be communicatively coupled to the app subnet(s) 1026 contained in the control plane app tier 1024 and an Internet gateway 1034 that can be contained in the control plane VCN 1016, and the app subnet(s) 1026 can be communicatively coupled to the DB subnet(s) 1030 contained in the control plane data tier 1028 and a service gateway 1036 and a network address translation (NAT) gateway 1038. The control plane VCN 1016 can include the service gateway 1036 and the NAT gateway 1038.

The control plane VCN 1016 can include a data plane mirror app tier 1040 that can include app subnet(s) 1026. The app subnet(s) 1026 contained in the data plane mirror app tier 1040 can include a virtual network interface controller (VNIC) 1042 that can execute a compute instance 1044. The compute instance 1044 can communicatively couple the app subnet(s) 1026 of the data plane mirror app tier 1040 to app subnet(s) 1026 that can be contained in a data plane app tier 1046.

The data plane VCN 1018 can include the data plane app tier 1046, a data plane DMZ tier 1048, and a data plane data tier 1050. The data plane DMZ tier 1048 can include LB subnet(s) 1022 that can be communicatively coupled to the app subnet(s) 1026 of the data plane app tier 1046 and the Internet gateway 1034 of the data plane VCN 1018. The app subnet(s) 1026 can be communicatively coupled to the service gateway 1036 of the data plane VCN 1018 and the NAT gateway 1038 of the data plane VCN 1018. The data plane data tier 1050 can also include the DB subnet(s) 1030 that can be communicatively coupled to the app subnet(s) 1026 of the data plane app tier 1046.

The Internet gateway 1034 of the control plane VCN 1016 and of the data plane VCN 1018 can be communicatively coupled to a metadata management service 1052 that can be communicatively coupled to public Internet 1054. Public Internet 1054 can be communicatively coupled to the NAT gateway 1038 of the control plane VCN 1016 and of the data plane VCN 1018. The service gateway 1036 of the control plane VCN 1016 and of the data plane VCN 1018 can be communicatively coupled to cloud services 1056.

In some examples, the service gateway 1036 of the control plane VCN 1016 or of the data plane VCN 1018 can make application programming interface (API) calls to cloud services 1056 without going through public Internet 1054. The API calls to cloud services 1056 from the service gateway 1036 can be one-way: the service gateway 1036 can make API calls to cloud services 1056, and cloud services 1056 can send requested data to the service gateway 1036. But, cloud services 1056 may not initiate API calls to the service gateway 1036.

In some examples, the secure host tenancy 1004 can be directly connected to the service tenancy 1019, which may be otherwise isolated. The secure host subnet 1008 can communicate with the SSH subnet 1014 through an LPG 1010 that may enable two-way communication over an otherwise isolated system. Connecting the secure host subnet 1008 to the SSH subnet 1014 may give the secure host subnet 1008 access to other entities within the service tenancy 1019.

The control plane VCN 1016 may allow users of the service tenancy 1019 to set up or otherwise provision desired resources. Desired resources provisioned in the control plane VCN 1016 may be deployed or otherwise used in the data plane VCN 1018. In some examples, the control plane VCN 1016 can be isolated from the data plane VCN 1018, and the data plane mirror app tier 1040 of the control plane VCN 1016 can communicate with the data plane app tier 1046 of the data plane VCN 1018 via VNICs 1042 that can be contained in the data plane mirror app tier 1040 and the data plane app tier 1046.

In some examples, users of the system, or customers, can make requests, for example create, read, update, or delete (CRUD) operations, through public Internet 1054 that can communicate the requests to the metadata management service 1052. The metadata management service 1052 can communicate the request to the control plane VCN 1016 through the Internet gateway 1034. The request can be received by the LB subnet(s) 1022 contained in the control plane DMZ tier 1020. The LB subnet(s) 1022 may determine that the request is valid, and in response to this determination, the LB subnet(s) 1022 can transmit the request to app subnet(s) 1026 contained in the control plane app tier 1024. If the request is validated and requires a call to public Internet 1054, the call to public Internet 1054 may be transmitted to the NAT gateway 1038 that can make the call to public Internet 1054. Metadata that may be desired to be stored by the request can be stored in the DB subnet(s) 1030.

In some examples, the data plane mirror app tier 1040 can facilitate direct communication between the control plane VCN 1016 and the data plane VCN 1018. For example, changes, updates, or other suitable modifications to configuration may be desired to be applied to the resources contained in the data plane VCN 1018. Via a VNIC 1042, the control plane VCN 1016 can directly communicate with, and can thereby execute the changes, updates, or other suitable modifications to configuration to, resources contained in the data plane VCN 1018.

In some embodiments, the control plane VCN 1016 and the data plane VCN 1018 can be contained in the service tenancy 1019. In this case, the user, or the customer, of the system may not own or operate either the control plane VCN 1016 or the data plane VCN 1018. Instead, the IaaS provider may own or operate the control plane VCN 1016 and the data plane VCN 1018, both of which may be contained in the service tenancy 1019. This embodiment can enable isolation of networks that may prevent users or customers from interacting with other users', or other customers', resources. Also, this embodiment may allow users or customers of the system to store databases privately without needing to rely on public Internet 1054, which may not have a desired level of threat prevention, for storage.

In other embodiments, the LB subnet(s) 1022 contained in the control plane VCN 1016 can be configured to receive a signal from the service gateway 1036. In this embodiment, the control plane VCN 1016 and the data plane VCN 1018 may be configured to be called by a customer of the IaaS provider without calling public Internet 1054. Customers of the IaaS provider may desire this embodiment since database(s) that the customers use may be controlled by the IaaS provider and may be stored on the service tenancy 1019, which may be isolated from public Internet 1054.

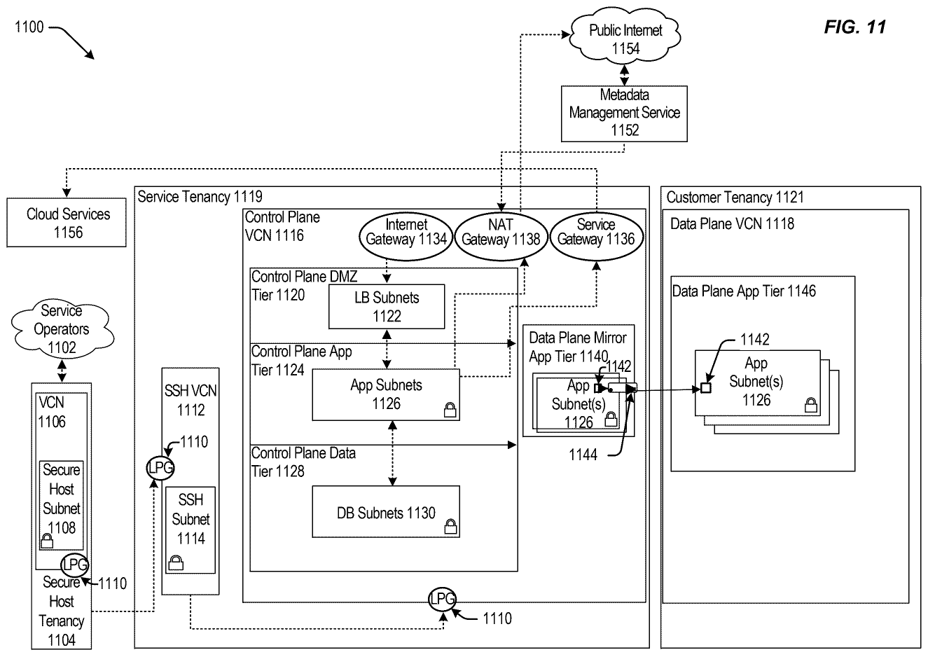

FIG. 11 is a block diagram 1100 illustrating another example pattern of an IaaS architecture, according to at least one embodiment. Service operators 1102 (e.g., service operators 1002 of FIG. 10) can be communicatively coupled to a secure host tenancy 1104 (e.g., the secure host tenancy 1004 of FIG. 10) that can include a virtual cloud network (VCN) 1106 (e.g., the VCN 1006 of FIG. 10) and a secure host subnet 1108 (e.g., the secure host subnet 1008 of FIG. 10). The VCN 1106 can include a local peering gateway (LPG) 1110 (e.g., the LPG 1010 of FIG. 10) that can be communicatively coupled to a secure shell (SSH) VCN 1112 (e.g., the SSH VCN 1012 of FIG. 10) via an LPG 1010 contained in the SSH VCN 1112. The SSH VCN 1112 can include an SSH subnet 1114 (e.g., the SSH subnet 1014 of FIG. 10), and the SSH VCN 1112 can be communicatively coupled to a control plane VCN 1116 (e.g., the control plane VCN 1016 of FIG. 10) via an LPG 1110 contained in the control plane VCN 1116. The control plane VCN 1116 can be contained in a service tenancy 1119 (e.g., the service tenancy 1019 of FIG. 10), and the data plane VCN 1118 (e.g., the data plane VCN 1018 of FIG. 10) can be contained in a customer tenancy 1121 that may be owned or operated by users, or customers, of the system.

The control plane VCN 1116 can include a control plane DMZ tier 1120 (e.g., the control plane DMZ tier 1020 of FIG. 10) that can include LB subnet(s) 1122 (e.g., LB subnet(s) 1022 of FIG. 10), a control plane app tier 1124 (e.g., the control plane app tier 1024 of FIG. 10) that can include app subnet(s) 1126 (e.g., app subnet(s) 1026 of FIG. 10), a control plane data tier 1128 (e.g., the control plane data tier 1028 of FIG. 10) that can include database (DB) subnet(s) 1130 (e.g., similar to DB subnet(s) 1030 of FIG. 10). The LB subnet(s) 1122 contained in the control plane DMZ tier 1120 can be communicatively coupled to the app subnet(s) 1126 contained in the control plane app tier 1124 and an Internet gateway 1134 (e.g., the Internet gateway 1034 of FIG. 10) that can be contained in the control plane VCN 1116, and the app subnet(s) 1126 can be communicatively coupled to the DB subnet(s) 1130 contained in the control plane data tier 1128 and a service gateway 1136 (e.g., the service gateway 1036 of FIG. 10) and a network address translation (NAT) gateway 1138 (e.g., the NAT gateway 1038 of FIG. 10). The control plane VCN 1116 can include the service gateway 1136 and the NAT gateway 1138.

The control plane VCN 1116 can include a data plane mirror app tier 1140 (e.g., the data plane mirror app tier 1040 of FIG. 10) that can include app subnet(s) 1126. The app subnet(s) 1126 contained in the data plane mirror app tier 1140 can include a virtual network interface controller (VNIC) 1142 (e.g., the VNIC of 1042) that can execute a compute instance 1144 (e.g., similar to the compute instance 1044 of FIG. 10). The compute instance 1144 can facilitate communication between the app subnet(s) 1126 of the data plane mirror app tier 1140 and the app subnet(s) 1126 that can be contained in a data plane app tier 1146 (e.g., the data plane app tier 1046 of FIG. 10) via the VNIC 1142 contained in the data plane mirror app tier 1140 and the VNIC 1142 contained in the data plane app tier 1146.

The Internet gateway 1134 contained in the control plane VCN 1116 can be communicatively coupled to a metadata management service 1152 (e.g., the metadata management service 1052 of FIG. 10) that can be communicatively coupled to public Internet 1154 (e.g., public Internet 1054 of FIG. 10). Public Internet 1154 can be communicatively coupled to the NAT gateway 1138 contained in the control plane VCN 1116. The service gateway 1136 contained in the control plane VCN 1116 can be communicatively coupled to cloud services 1156 (e.g., cloud services 1056 of FIG. 10).

In some examples, the data plane VCN 1118 can be contained in the customer tenancy 1121. In this case, the IaaS provider may provide the control plane VCN 1116 for each customer, and the IaaS provider may, for each customer, set up a unique compute instance 1144 that is contained in the service tenancy 1119. Each compute instance 1144 may allow communication between the control plane VCN 1116, contained in the service tenancy 1119, and the data plane VCN 1118 that is contained in the customer tenancy 1121. The compute instance 1144 may allow resources, that are provisioned in the control plane VCN 1116 that is contained in the service tenancy 1119, to be deployed or otherwise used in the data plane VCN 1118 that is contained in the customer tenancy 1121.

In other examples, the customer of the IaaS provider may have databases that live in the customer tenancy 1121. In this example, the control plane VCN 1116 can include the data plane mirror app tier 1140 that can include app subnet(s) 1126. The data plane mirror app tier 1140 can reside in the data plane VCN 1118, but the data plane mirror app tier 1140 may not live in the data plane VCN 1118. That is, the data plane mirror app tier 1140 may have access to the customer tenancy 1121, but the data plane mirror app tier 1140 may not exist in the data plane VCN 1118 or be owned or operated by the customer of the IaaS provider. The data plane mirror app tier 1140 may be configured to make calls to the data plane VCN 1118 but may not be configured to make calls to any entity contained in the control plane VCN 1116. The customer may desire to deploy or otherwise use resources in the data plane VCN 1118 that are provisioned in the control plane VCN 1116, and the data plane mirror app tier 1140 can facilitate the desired deployment, or other usage of resources, of the customer.

In some embodiments, the customer of the IaaS provider can apply filters to the data plane VCN 1118. In this embodiment, the customer can determine what the data plane VCN 1118 can access, and the customer may restrict access to public Internet 1154 from the data plane VCN 1118. The IaaS provider may not be able to apply filters or otherwise control access of the data plane VCN 1118 to any outside networks or databases. Applying filters and controls by the customer onto the data plane VCN 1118, contained in the customer tenancy 1121, can help isolate the data plane VCN 1118 from other customers and from public Internet 1154.

In some embodiments, cloud services 1156 can be called by the service gateway 1136 to access services that may not exist on public Internet 1154, on the control plane VCN 1116, or on the data plane VCN 1118. The connection between cloud services 1156 and the control plane VCN 1116 or the data plane VCN 1118 may not be live or continuous. Cloud services 1156 may exist on a different network owned or operated by the IaaS provider. Cloud services 1156 may be configured to receive calls from the service gateway 1136 and may be configured to not receive calls from public Internet 1154. Some cloud services 1156 may be isolated from other cloud services 1156, and the control plane VCN 1116 may be isolated from cloud services 1156 that may not be in the same region as the control plane VCN 1116. For example, the control plane VCN 1116 may be located in “Region 1,” and cloud service “Deployment 10,” may be located in Region 1 and in “Region 2.” If a call to Deployment 10 is made by the service gateway 1136 contained in the control plane VCN 1116 located in Region 1, the call may be transmitted to Deployment 10 in Region 1. In this example, the control plane VCN 1116, or Deployment 10 in Region 1, may not be communicatively coupled to, or otherwise in communication with, Deployment 10 in Region 2.

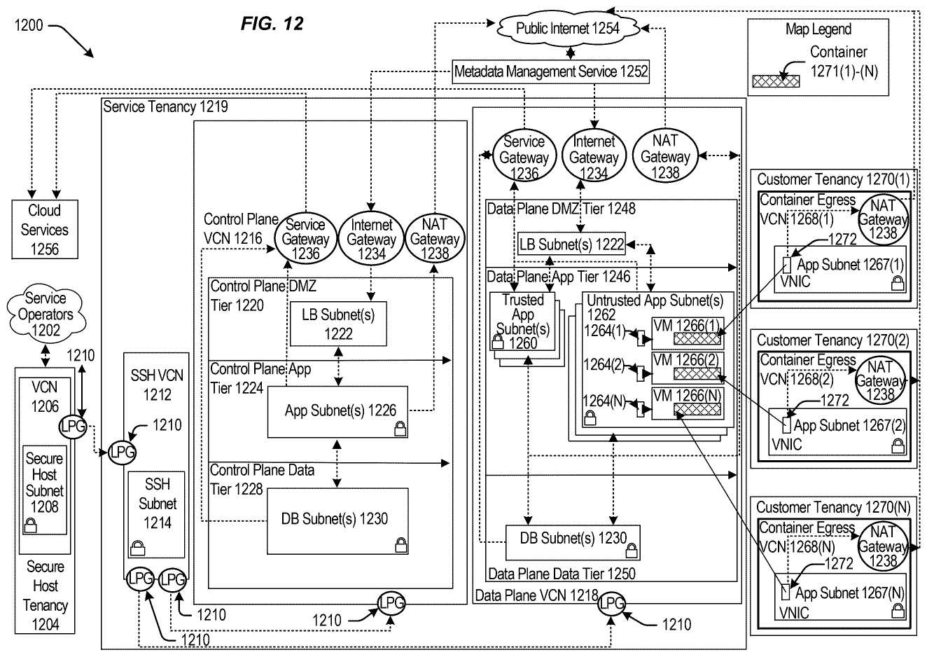

FIG. 12 is a block diagram 1200 illustrating another example pattern of an IaaS architecture, according to at least one embodiment. Service operators 1202 (e.g., service operators 1002 of FIG. 10) can be communicatively coupled to a secure host tenancy 1204 (e.g., the secure host tenancy 1004 of FIG. 10) that can include a virtual cloud network (VCN) 1206 (e.g., the VCN 1006 of FIG. 10) and a secure host subnet 1208 (e.g., the secure host subnet 1008 of FIG. 10). The VCN 1206 can include an LPG 1210 (e.g., the LPG 1010 of FIG. 10) that can be communicatively coupled to an SSH VCN 1212 (e.g., the SSH VCN 1012 of FIG. 10) via an LPG 1210 contained in the SSH VCN 1212. The SSH VCN 1212 can include an SSH subnet 1214 (e.g., the SSH subnet 1014 of FIG. 10), and the SSH VCN 1212 can be communicatively coupled to a control plane VCN 1216 (e.g., the control plane VCN 1016 of FIG. 10) via an LPG 1210 contained in the control plane VCN 1216 and to a data plane VCN 1218 (e.g., the data plane 1018 of FIG. 10) via an LPG 1210 contained in the data plane VCN 1218. The control plane VCN 1216 and the data plane VCN 1218 can be contained in a service tenancy 1219 (e.g., the service tenancy 1019 of FIG. 10).

The control plane VCN 1216 can include a control plane DMZ tier 1220 (e.g., the control plane DMZ tier 1020 of FIG. 10) that can include load balancer (LB) subnet(s) 1222 (e.g., LB subnet(s) 1022 of FIG. 10), a control plane app tier 1224 (e.g., the control plane app tier 1024 of FIG. 10) that can include app subnet(s) 1226 (e.g., similar to app subnet(s) 1026 of FIG. 10), a control plane data tier 1228 (e.g., the control plane data tier 1028 of FIG. 10) that can include DB subnet(s) 1230. The LB subnet(s) 1222 contained in the control plane DMZ tier 1220 can be communicatively coupled to the app subnet(s) 1226 contained in the control plane app tier 1224 and to an Internet gateway 1234 (e.g., the Internet gateway 1034 of FIG. 10) that can be contained in the control plane VCN 1216, and the app subnet(s) 1226 can be communicatively coupled to the DB subnet(s) 1230 contained in the control plane data tier 1228 and to a service gateway 1236 (e.g., the service gateway of FIG. 10) and a network address translation (NAT) gateway 1238 (e.g., the NAT gateway 1038 of FIG. 10). The control plane VCN 1216 can include the service gateway 1236 and the NAT gateway 1238.

The data plane VCN 1218 can include a data plane app tier 1246 (e.g., the data plane app tier 1046 of FIG. 10), a data plane DMZ tier 1248 (e.g., the data plane DMZ tier 1048 of FIG. 10), and a data plane data tier 1250 (e.g., the data plane data tier 1050 of FIG. 10). The data plane DMZ tier 1248 can include LB subnet(s) 1222 that can be communicatively coupled to trusted app subnet(s) 1260 and untrusted app subnet(s) 1262 of the data plane app tier 1246 and the Internet gateway 1234 contained in the data plane VCN 1218. The trusted app subnet(s) 1260 can be communicatively coupled to the service gateway 1236 contained in the data plane VCN 1218, the NAT gateway 1238 contained in the data plane VCN 1218, and DB subnet(s) 1230 contained in the data plane data tier 1250. The untrusted app subnet(s) 1262 can be communicatively coupled to the service gateway 1236 contained in the data plane VCN 1218 and DB subnet(s) 1230 contained in the data plane data tier 1250. The data plane data tier 1250 can include DB subnet(s) 1230 that can be communicatively coupled to the service gateway 1236 contained in the data plane VCN 1218.

The untrusted app subnet(s) 1262 can include one or more primary VNICs 1264(1)-(N) that can be communicatively coupled to tenant virtual machines (VMs) 1266(1)-(N). Each tenant VM 1266(1)-(N) can be communicatively coupled to a respective app subnet 1267(1)-(N) that can be contained in respective container egress VCNs 1268(1)-(N) that can be contained in respective customer tenancies 1270(1)-(N). Respective secondary VNICs 1272(1)-(N) can facilitate communication between the untrusted app subnet(s) 1262 contained in the data plane VCN 1218 and the app subnet contained in the container egress VCNs 1268(1)-(N). Each container egress VCNs 1268(1)-(N) can include a NAT gateway 1238 that can be communicatively coupled to public Internet 1254 (e.g., public Internet 1054 of FIG. 10).

The Internet gateway 1234 contained in the control plane VCN 1216 and contained in the data plane VCN 1218 can be communicatively coupled to a metadata management service 1252 (e.g., the metadata management system 1052 of FIG. 10) that can be communicatively coupled to public Internet 1254. Public Internet 1254 can be communicatively coupled to the NAT gateway 1238 contained in the control plane VCN 1216 and contained in the data plane VCN 1218. The service gateway 1236 contained in the control plane VCN 1216 and contained in the data plane VCN 1218 can be communicatively coupled to cloud services 1256.

In some embodiments, the data plane VCN 1218 can be integrated with customer tenancies 1270. This integration can be useful or desirable for customers of the IaaS provider in some cases such as a case that may desire support when executing code. The customer may provide code to run that may be destructive, may communicate with other customer resources, or may otherwise cause undesirable effects. In response to this, the IaaS provider may determine whether to run code given to the IaaS provider by the customer.

In some examples, the customer of the IaaS provider may grant temporary network access to the IaaS provider and request a function to be attached to the data plane app tier 1246. Code to run the function may be executed in the VMs 1266(1)-(N), and the code may not be configured to run anywhere else on the data plane VCN 1218. Each VM 1266(1)-(N) may be connected to one customer tenancy 1270. Respective containers 1271(1)-(N) contained in the VMs 1266(1)-(N) may be configured to run the code. In this case, there can be a dual isolation (e.g., the containers 1271(1)-(N) running code, where the containers 1271(1)-(N) may be contained in at least the VM 1266(1)-(N) that are contained in the untrusted app subnet(s) 1262), which may help prevent incorrect or otherwise undesirable code from damaging the network of the IaaS provider or from damaging a network of a different customer. The containers 1271(1)-(N) may be communicatively coupled to the customer tenancy 1270 and may be configured to transmit or receive data from the customer tenancy 1270. The containers 1271(1)-(N) may not be configured to transmit or receive data from any other entity in the data plane VCN 1218. Upon completion of running the code, the IaaS provider may kill or otherwise dispose of the containers 1271(1)-(N).

In some embodiments, the trusted app subnet(s) 1260 may run code that may be owned or operated by the IaaS provider. In this embodiment, the trusted app subnet(s) 1260 may be communicatively coupled to the DB subnet(s) 1230 and be configured to execute CRUD operations in the DB subnet(s) 1230. The untrusted app subnet(s) 1262 may be communicatively coupled to the DB subnet(s) 1230, but in this embodiment, the untrusted app subnet(s) may be configured to execute read operations in the DB subnet(s) 1230. The containers 1271(1)-(N) that can be contained in the VM 1266(1)-(N) of each customer and that may run code from the customer may not be communicatively coupled with the DB subnet(s) 1230.

In other embodiments, the control plane VCN 1216 and the data plane VCN 1218 may not be directly communicatively coupled. In this embodiment, there may be no direct communication between the control plane VCN 1216 and the data plane VCN 1218. However, communication can occur indirectly through at least one method. An LPG 1210 may be established by the IaaS provider that can facilitate communication between the control plane VCN 1216 and the data plane VCN 1218. In another example, the control plane VCN 1216 or the data plane VCN 1218 can make a call to cloud services 1256 via the service gateway 1236. For example, a call to cloud services 1256 from the control plane VCN 1216 can include a request for a service that can communicate with the data plane VCN 1218.

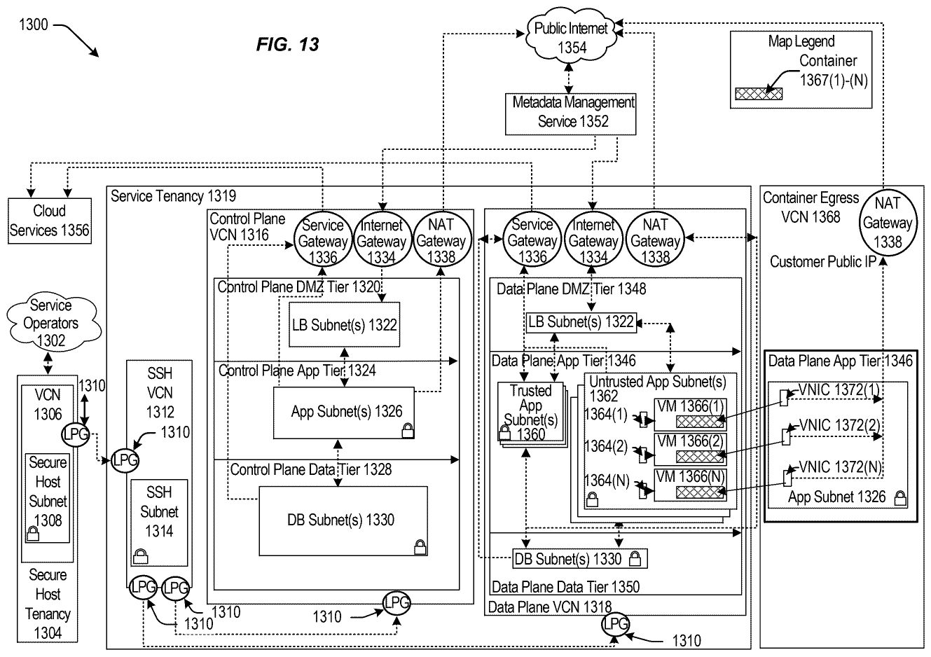

FIG. 13 is a block diagram 1300 illustrating another example pattern of an IaaS architecture, according to at least one embodiment. Service operators 1302 (e.g., service operators 1002 of FIG. 10) can be communicatively coupled to a secure host tenancy 1304 (e.g., the secure host tenancy 1004 of FIG. 10) that can include a virtual cloud network (VCN) 1306 (e.g., the VCN 1006 of FIG. 10) and a secure host subnet 1308 (e.g., the secure host subnet 1008 of FIG. 10). The VCN 1306 can include an LPG 1310 (e.g., the LPG 1010 of FIG. 10) that can be communicatively coupled to an SSH VCN 1312 (e.g., the SSH VCN 1012 of FIG. 10) via an LPG 1310 contained in the SSH VCN 1312. The SSH VCN 1312 can include an SSH subnet 1314 (e.g., the SSH subnet 1014 of FIG. 10), and the SSH VCN 1312 can be communicatively coupled to a control plane VCN 1316 (e.g., the control plane VCN 1016 of FIG. 10) via an LPG 1310 contained in the control plane VCN 1316 and to a data plane VCN 1318 (e.g., the data plane 1018 of FIG. 10) via an LPG 1310 contained in the data plane VCN 1318. The control plane VCN 1316 and the data plane VCN 1318 can be contained in a service tenancy 1319 (e.g., the service tenancy 1019 of FIG. 10).

The control plane VCN 1316 can include a control plane DMZ tier 1320 (e.g., the control plane DMZ tier 1020 of FIG. 10) that can include LB subnet(s) 1322 (e.g., LB subnet(s) 1022 of FIG. 10), a control plane app tier 1324 (e.g., the control plane app tier 1024 of FIG. 10) that can include app subnet(s) 1326 (e.g., app subnet(s) 1026 of FIG. 10), a control plane data tier 1328 (e.g., the control plane data tier 1028 of FIG. 10) that can include DB subnet(s) 1330 (e.g., DB subnet(s) 1230 of FIG. 12). The LB subnet(s) 1322 contained in the control plane DMZ tier 1320 can be communicatively coupled to the app subnet(s) 1326 contained in the control plane app tier 1324 and to an Internet gateway 1334 (e.g., the Internet gateway 1034 of FIG. 10) that can be contained in the control plane VCN 1316, and the app subnet(s) 1326 can be communicatively coupled to the DB subnet(s) 1330 contained in the control plane data tier 1328 and to a service gateway 1336 (e.g., the service gateway of FIG. 10) and a network address translation (NAT) gateway 1338 (e.g., the NAT gateway 1038 of FIG. 10). The control plane VCN 1316 can include the service gateway 1336 and the NAT gateway 1338.

The data plane VCN 1318 can include a data plane app tier 1346 (e.g., the data plane app tier 1046 of FIG. 10), a data plane DMZ tier 1348 (e.g., the data plane DMZ tier 1048 of FIG. 10), and a data plane data tier 1350 (e.g., the data plane data tier 1050 of FIG. 10). The data plane DMZ tier 1348 can include LB subnet(s) 1322 that can be communicatively coupled to trusted app subnet(s) 1360 (e.g., trusted app subnet(s) 1260 of FIG. 12) and untrusted app subnet(s) 1362 (e.g., untrusted app subnet(s) 1262 of FIG. 12) of the data plane app tier 1346 and the Internet gateway 1334 contained in the data plane VCN 1318. The trusted app subnet(s) 1360 can be communicatively coupled to the service gateway 1336 contained in the data plane VCN 1318, the NAT gateway 1338 contained in the data plane VCN 1318, and DB subnet(s) 1330 contained in the data plane data tier 1350. The untrusted app subnet(s) 1362 can be communicatively coupled to the service gateway 1336 contained in the data plane VCN 1318 and DB subnet(s) 1330 contained in the data plane data tier 1350. The data plane data tier 1350 can include DB subnet(s) 1330 that can be communicatively coupled to the service gateway 1336 contained in the data plane VCN 1318.

The untrusted app subnet(s) 1362 can include primary VNICs 1364(1)-(N) that can be communicatively coupled to tenant virtual machines (VMs) 1366(1)-(N) residing within the untrusted app subnet(s) 1362. Each tenant VM 1366(1)-(N) can run code in a respective container 1367(1)-(N), and be communicatively coupled to an app subnet 1326 that can be contained in a data plane app tier 1346 that can be contained in a container egress VCN 1368. Respective secondary VNICs 1372(1)-(N) can facilitate communication between the untrusted app subnet(s) 1362 contained in the data plane VCN 1318 and the app subnet contained in the container egress VCN 1368. The container egress VCN can include a NAT gateway 1338 that can be communicatively coupled to public Internet 1354 (e.g., public Internet 1054 of FIG. 10).

The Internet gateway 1334 contained in the control plane VCN 1316 and contained in the data plane VCN 1318 can be communicatively coupled to a metadata management service 1352 (e.g., the metadata management system 1052 of FIG. 10) that can be communicatively coupled to public Internet 1354. Public Internet 1354 can be communicatively coupled to the NAT gateway 1338 contained in the control plane VCN 1316 and contained in the data plane VCN 1318. The service gateway 1336 contained in the control plane VCN 1316 and contained in the data plane VCN 1318 can be communicatively coupled to cloud services 1356.

In some examples, the pattern illustrated by the architecture of block diagram 1300 of FIG. 13 may be considered an exception to the pattern illustrated by the architecture of block diagram 1200 of FIG. 12 and may be desirable for a customer of the IaaS provider if the IaaS provider cannot directly communicate with the customer (e.g., a disconnected region). The respective containers 1367(1)-(N) that are contained in the VMs 1366(1)-(N) for each customer can be accessed in real-time by the customer. The containers 1367(1)-(N) may be configured to make calls to respective secondary VNICs 1372(1)-(N) contained in app subnet(s) 1326 of the data plane app tier 1346 that can be contained in the container egress VCN 1368. The secondary VNICs 1372(1)-(N) can transmit the calls to the NAT gateway 1338 that may transmit the calls to public Internet 1354. In this example, the containers 1367(1)-(N) that can be accessed in real-time by the customer can be isolated from the control plane VCN 1316 and can be isolated from other entities contained in the data plane VCN 1318. The containers 1367(1)-(N) may also be isolated from resources from other customers.

In other examples, the customer can use the containers 1367(1)-(N) to call cloud services 1356. In this example, the customer may run code in the containers 1367(1)-(N) that requests a service from cloud services 1356. The containers 1367(1)-(N) can transmit this request to the secondary VNICs 1372(1)-(N) that can transmit the request to the NAT gateway that can transmit the request to public Internet 1354. Public Internet 1354 can transmit the request to LB subnet(s) 1322 contained in the control plane VCN 1316 via the Internet gateway 1334. In response to determining the request is valid, the LB subnet(s) can transmit the request to app subnet(s) 1326 that can transmit the request to cloud services 1356 via the service gateway 1336.

It should be appreciated that IaaS architectures 1000, 1100, 1200, 1300 depicted in the figures may have other components than those depicted. Further, the embodiments shown in the figures are only some examples of a cloud infrastructure system that may incorporate an embodiment of the disclosure. In some other embodiments, the IaaS systems may have more or fewer components than shown in the figures, may combine two or more components, or may have a different configuration or arrangement of components.

In certain embodiments, the IaaS systems described herein may include a suite of applications, middleware, and database service offerings that are delivered to a customer in a self-service, subscription-based, elastically scalable, reliable, highly available, and secure manner. An example of such an IaaS system is the Oracle Cloud Infrastructure (OCI) provided by the present assignee.

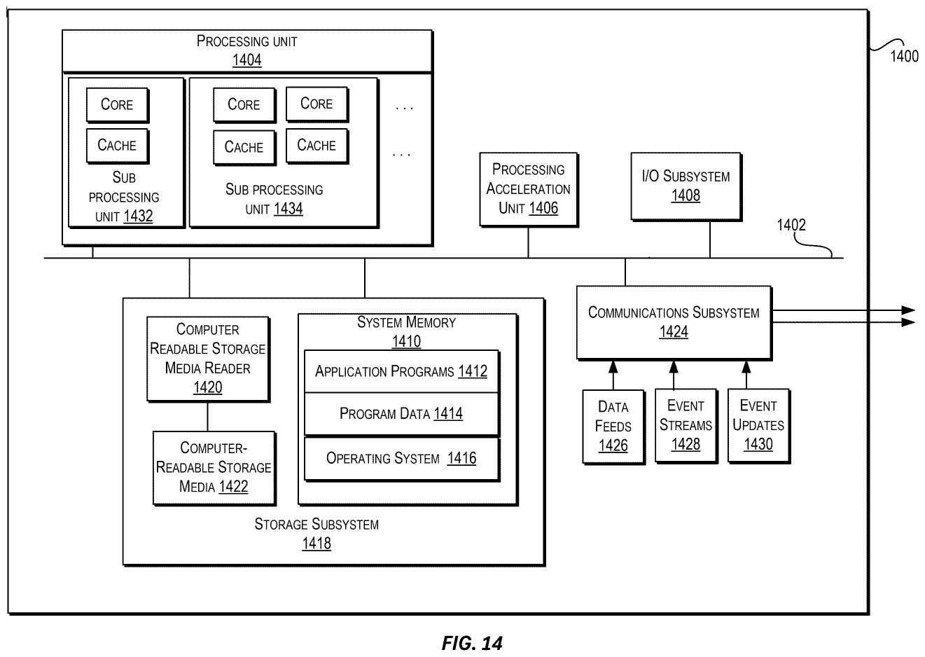

FIG. 14 illustrates an example computer system 1400, in which various embodiments may be implemented. The system 1400 may be used to implement any of the computer systems described above. As shown in the figure, computer system 1400 includes a processing unit 1404 that communicates with a number of peripheral subsystems via a bus subsystem 1402. These peripheral subsystems may include a processing acceleration unit 1406, an I/O subsystem 1408, a storage subsystem 1418 and a communications subsystem 1424. Storage subsystem 1418 includes tangible computer-readable storage media 1422 and a system memory 1410.

Bus subsystem 1402 provides a mechanism for letting the various components and subsystems of computer system 1400 communicate with each other as intended. Although bus subsystem 1402 is shown schematically as a single bus, alternative embodiments of the bus subsystem may utilize multiple buses. Bus subsystem 1402 may be any of several types of bus structures including a memory bus or memory controller, a peripheral bus, and a local bus using any of a variety of bus architectures. For example, such architectures may include an Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, which can be implemented as a Mezzanine bus manufactured to the IEEE P1386.1 standard.

Processing unit 1404, which can be implemented as one or more integrated circuits (e.g., a conventional microprocessor or microcontroller), controls the operation of computer system 1400. One or more processors may be included in processing unit 1404. These processors may include single core or multicore processors. In certain embodiments, processing unit 1404 may be implemented as one or more independent processing units 1432 and/or 1434 with single or multicore processors included in each processing unit. In other embodiments, processing unit 1404 may also be implemented as a quad-core processing unit formed by integrating two dual-core processors into a single chip.

In various embodiments, processing unit 1404 can execute a variety of programs in response to program code and can maintain multiple concurrently executing programs or processes. At any given time, some or all of the program code to be executed can be resident in processor(s) 1404 and/or in storage subsystem 1418. Through suitable programming, processor(s) 1404 can provide various functionalities described above. Computer system 1400 may additionally include a processing acceleration unit 1406, which can include a digital signal processor (DSP), a special-purpose processor, and/or the like.

I/O subsystem 1408 may include user interface input devices and user interface output devices. User interface input devices may include a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may include, for example, motion sensing and/or gesture recognition devices such as the Microsoft Kinect® motion sensor that enables users to control and interact with an input device, such as the Microsoft Xbox® 360 game controller, through a natural user interface using gestures and spoken commands. User interface input devices may also include eye gesture recognition devices such as the Google Glass® blink detector that detects eye activity (e.g., ‘blinking’ while taking pictures and/or making a menu selection) from users and transforms the eye gestures as input into an input device (e.g., Google Glass®). Additionally, user interface input devices may include voice recognition sensing devices that enable users to interact with voice recognition systems (e.g., Siri® navigator), through voice commands.

User interface input devices may also include, without limitation, three dimensional (3D) mice, joysticks or pointing sticks, gamepads and graphic tablets, and audio/visual devices such as speakers, digital cameras, digital camcorders, portable media players, webcams, image scanners, fingerprint scanners, barcode reader 3D scanners, 3D printers, laser rangefinders, and eye gaze tracking devices. Additionally, user interface input devices may include, for example, medical imaging input devices such as computed tomography, magnetic resonance imaging, position emission tomography, medical ultrasonography devices. User interface input devices may also include, for example, audio input devices such as MIDI keyboards, digital musical instruments and the like.

User interface output devices may include a display subsystem, indicator lights, or non-visual displays such as audio output devices, etc. The display subsystem may be a cathode ray tube (CRT), a flat-panel device, such as that using a liquid crystal display (LCD) or plasma display, a projection device, a touch screen, and the like. In general, use of the term “output device” is intended to include all possible types of devices and mechanisms for outputting information from computer system 1400 to a user or other computer. For example, user interface output devices may include, without limitation, a variety of display devices that visually convey text, graphics and audio/video information such as monitors, printers, speakers, headphones, automotive navigation systems, plotters, voice output devices, and modems.

Computer system 1400 may comprise a storage subsystem 1418 that provides a tangible non-transitory computer-readable storage medium for storing software and data constructs that provide the functionality of the embodiments described in this disclosure. The software can include programs, code modules, instructions, scripts, etc., that when executed by one or more cores or processors of processing unit 1404 provide the functionality described above. Storage subsystem 1418 may also provide a repository for storing data used in accordance with the present disclosure.

As depicted in the example in FIG. 14, storage subsystem 1418 can include various components including a system memory 1410, computer-readable storage media 1422, and a computer readable storage media reader 1420. System memory 1410 may store program instructions that are loadable and executable by processing unit 1404. System memory 1410 may also store data that is used during the execution of the instructions and/or data that is generated during the execution of the program instructions. Various different kinds of programs may be loaded into system memory 1410 including but not limited to client applications, Web browsers, mid-tier applications, relational database management systems (RDBMS), virtual machines, containers, etc.

System memory 1410 may also store an operating system 1416. Examples of operating system 1416 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome® OS, and the like) and/or mobile operating systems such as iOS, Windows® Phone, Android® OS, BlackBerry® OS, and Palm® OS operating systems. In certain implementations where computer system 1400 executes one or more virtual machines, the virtual machines along with their guest operating systems (GOSs) may be loaded into system memory 1410 and executed by one or more processors or cores of processing unit 1404.

System memory 1410 can come in different configurations depending upon the type of computer system 1400. For example, system memory 1410 may be volatile memory (such as random access memory (RAM)) and/or non-volatile memory (such as read-only memory (ROM), flash memory, etc.) Different types of RAM configurations may be provided including a static random access memory (SRAM), a dynamic random access memory (DRAM), and others. In some implementations, system memory 1410 may include a basic input/output system (BIOS) containing basic routines that help to transfer information between elements within computer system 1400, such as during start-up.

Computer-readable storage media 1422 may represent remote, local, fixed, and/or removable storage devices plus storage media for temporarily and/or more permanently containing, storing, computer-readable information for use by computer system 1400 including instructions executable by processing unit 1404 of computer system 1400.

Computer-readable storage media 1422 can include any appropriate media known or used in the art, including storage media and communication media, such as but not limited to, volatile and non-volatile, removable and non-removable media implemented in any method or technology for storage and/or transmission of information. This can include tangible computer-readable storage media such as RAM, ROM, electronically erasable programmable ROM (EEPROM), flash memory or other memory technology, CD-ROM, digital versatile disk (DVD), or other optical storage, magnetic cassettes, magnetic tape, magnetic disk storage or other magnetic storage devices, or other tangible computer readable media.

By way of example, computer-readable storage media 1422 may include a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM, DVD, and Blu-Ray® disk, or other optical media. Computer-readable storage media 1422 may include, but is not limited to, Zip® drives, flash memory cards, universal serial bus (USB) flash drives, secure digital (SD) cards, DVD disks, digital video tape, and the like. Computer-readable storage media 1422 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, DRAM-based SSDs, magnetoresistive RAM (MRAM) SSDs, and hybrid SSDs that use a combination of DRAM and flash memory based SSDs. The disk drives and their associated computer-readable media may provide non-volatile storage of computer-readable instructions, data structures, program modules, and other data for computer system 1400.

Machine-readable instructions executable by one or more processors or cores of processing unit 1404 may be stored on a non-transitory computer-readable storage medium. A non-transitory computer-readable storage medium can include physically tangible memory or storage devices that include volatile memory storage devices and/or non-volatile storage devices. Examples of non-transitory computer-readable storage medium include magnetic storage media (e.g., disk or tapes), optical storage media (e.g., DVDs, CDs), various types of RAM, ROM, or flash memory, hard drives, floppy drives, detachable memory drives (e.g., USB drives), or other type of storage device.

Communications subsystem 1424 provides an interface to other computer systems and networks. Communications subsystem 1424 serves as an interface for receiving data from and transmitting data to other systems from computer system 1400. For example, communications subsystem 1424 may enable computer system 1400 to connect to one or more devices via the Internet. In some embodiments communications subsystem 1424 can include radio frequency (RF) transceiver components for accessing wireless voice and/or data networks (e.g., using cellular telephone technology, advanced data network technology, such as 3G, 4G or EDGE (enhanced data rates for global evolution), WiFi (IEEE 902.11 family standards, or other mobile communication technologies, or any combination thereof)), global positioning system (GPS) receiver components, and/or other components. In some embodiments communications subsystem 1424 can provide wired network connectivity (e.g., Ethernet) in addition to or instead of a wireless interface.

In some embodiments, communications subsystem 1424 may also receive input communication in the form of structured and/or unstructured data feeds 1426, event streams 1428, event updates 1430, and the like on behalf of one or more users who may use computer system 1400.

By way of example, communications subsystem 1424 may be configured to receive data feeds 1426 in real-time from users of social networks and/or other communication services such as Twitter® feeds, Facebook® updates, web feeds such as Rich Site Summary (RSS) feeds, and/or real-time updates from one or more third party information sources.

Additionally, communications subsystem 1424 may also be configured to receive data in the form of continuous data streams, which may include event streams 1428 of real-time events and/or event updates 1430, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 1424 may also be configured to output the structured and/or unstructured data feeds 1426, event streams 1428, event updates 1430, and the like to one or more databases that may be in communication with one or more streaming data source computers coupled to computer system 1400.

Computer system 1400 can be one of various types, including a handheld portable device (e.g., an iPhone® cellular phone, an iPad® computing tablet, a PDA), a wearable device (e.g., a Google Glass® head mounted display), a PC, a workstation, a mainframe, a kiosk, a server rack, or any other data processing system.

Due to the ever-changing nature of computers and networks, the description of computer system 1400 depicted in the figure is intended only as a specific example. Many other configurations having more or fewer components than the system depicted in the figure are possible. For example, customized hardware might also be used and/or particular elements might be implemented in hardware, firmware, software (including applets), or a combination. Further, connection to other computing devices, such as network input/output devices, may be employed. Based on the disclosure and teachings provided herein, a person of ordinary skill in the art will appreciate other ways and/or methods to implement the various embodiments.

Although specific embodiments have been described, various modifications, alterations, alternative constructions, and equivalents are also encompassed within the scope of the disclosure. Embodiments are not restricted to operation within certain specific data processing environments, but are free to operate within a plurality of data processing environments. Additionally, although embodiments have been described using a particular series of transactions and steps, it should be apparent to those skilled in the art that the scope of the present disclosure is not limited to the described series of transactions and steps. Various features and aspects of the above-described embodiments may be used individually or jointly.

Further, while embodiments have been described using a particular combination of hardware and software, it should be recognized that other combinations of hardware and software are also within the scope of the present disclosure. Embodiments may be implemented only in hardware, or only in software, or using combinations thereof. The various processes described herein can be implemented on the same processor or different processors in any combination. Accordingly, where components or services are described as being configured to perform certain operations, such configuration can be accomplished, e.g., by designing electronic circuits to perform the operation, by programming programmable electronic circuits (such as microprocessors) to perform the operation, or any combination thereof. Processes can communicate using a variety of techniques including but not limited to conventional techniques for inter process communication, and different pairs of processes may use different techniques, or the same pair of processes may use different techniques at different times.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific disclosure embodiments have been described, these are not intended to be limiting. Various modifications and equivalents are within the scope of the following claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Preferred embodiments of this disclosure are described herein, including the best mode known for carrying out the disclosure. Variations of those preferred embodiments may become apparent to those of ordinary skill in the art upon reading the foregoing description. Those of ordinary skill should be able to employ such variations as appropriate and the disclosure may be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein.

All references, including publications, patent applications, and patents, cited herein are hereby incorporated by reference to the same extent as if each reference were individually and specifically indicated to be incorporated by reference and were set forth in its entirety herein.

In the foregoing specification, aspects of the disclosure are described with reference to specific embodiments thereof, but those skilled in the art will recognize that the disclosure is not limited thereto. Various features and aspects of the above-described disclosure may be used individually or jointly. Further, embodiments can be utilized in any number of environments and applications beyond those described herein without departing from the broader spirit and scope of the specification. The specification and drawings are, accordingly, to be regarded as illustrative rather than restrictive.

What is claimed is:

1. A computer-implemented method comprising:
receiving, by a computer system, a query in natural language;
generating, by the computer system, an input for a large language model, the input comprising a prompt generated based on the query;
identifying, by the computer system, a plurality of slots associated with a plurality of sections of a content item;
generating, by the large language model, a query result based on the input, the query result comprising a subset of slots of the plurality of slots selected in accordance with the plurality of sections;
extracting, by the computer system, one or more document chunks from a database storing a plurality of document chunks as one or more relevant document chunks associated with the query result;
formatting, by the computer system, the one or more relevant document chunks into a response to the query; and
providing, by the computer system, the response to a client system,
wherein the plurality of document chunks is generated by dividing each content item of a plurality of content items into the plurality of document chunks based on sections within each content item of the plurality of content items.

2. The computer-implemented method of claim 1, wherein the plurality of slots comprises at least one of medical named entity recognitions (NERs), keywords identified as frequently arising in clinical patient encounters, questions identified as frequently arising in clinical patient encounters, and information identified as frequently requested during clinical patient encounters.

3. The computer-implemented method of claim 1, wherein the document chunks in the database are produced by performing at least one of sliding window chunking and semantic chunking.

4. The computer-implemented method of claim 1, wherein extracting one or more document chunks from the database storing the plurality of document chunks further comprises extracting metadata associated with the one or more relevant document chunks.

5. The computer-implemented method of claim 4, wherein the metadata comprises at least one of a date of creation of a note, a note type, a note section, a specialist note, patient information, and a practitioner role.

6. The computer-implemented method of claim 4, wherein extracting the one or more document chunks from the database comprises using the metadata to determine a relevance of the one or more of the plurality of document chunks into the query.

7. The computer-implemented method of claim 1, wherein extracting one or more document chunks from the database further comprises:
identifying a time-dependent aspect in the query, and
selecting one or more of the plurality of document chunks based on the time-dependent aspect in the query.

8. The computer-implemented method of claim 1, wherein extracting one or more document chunks from the database further comprises performing at least one of a keyword search and a k-Nearest Neighbor (KNN) search on the plurality of document chunks in the database.

9. The computer-implemented method of claim 1, further comprising
enriching, by the computer system, at least one of the plurality of sections and the plurality of document chunks with embeddings.

10. The computer-implemented method of claim 9, wherein the embeddings comprise at least one of subsection header names and vector embeddings.

11. The computer-implemented method of claim 1, wherein formatting the one or more relevant document chunks into the response to the query comprises ranking the one or more relevant document chunks based on a predefined set of parameters.

12. A system comprising:
a computer system comprising one or more processors and memory storing computer executable instructions that, when executed by the one or more processors, configure the computer system to at least:
receive a query in natural language;
generate an input for a large language model, the input comprising a prompt generated based on the query;
identify a plurality of slots associated with a plurality of sections of a content item;
generating, by the large language model, a query result based on the input, the query result comprising a subset of slots of the plurality of slots selected in accordance with the plurality of sections;
extract one or more document chunks from a database storing a plurality of document chunks as one or more relevant document chunks associated with the query result;
format the one or more relevant document chunks into a response to the query; and
provide the response to the query to a client system,
wherein the plurality of document chunks are generated by dividing each document of a plurality of documents into the plurality of document chunks based on sections within each content item of a plurality of content items.

13. The system of claim 12, wherein the plurality of slots comprises at least one of medical named entity recognitions (NERs), keywords identified as frequently arising in clinical patient encounters, questions identified as frequently arising in clinical patient encounters, and information identified as frequently requested during clinical patient encounters.

14. The system of claim 12, wherein the document chunks in the database are produced by performing at least one of sliding window chunking and semantic chunking.

15. The system of claim 12, wherein extracting one or more document chunks from the database storing the plurality of document chunks further comprises extracting metadata associated with the one or more relevant document chunks.

16. The system of claim 15, wherein the metadata comprises at least one of a date of creation of a note, a note type, a note section, a specialist note, patient information, and a practitioner role.

17. The system of claim 15, wherein extracting the one or more document chunks from the database comprises using the metadata to determine a relevance of the one or more of the plurality of document chunks into the query.

18. The system of claim 12, wherein extracting one or more document chunks from the database further comprises
identifying a time-dependent aspect in the query, and
selecting one or more of the plurality of document chunks based on the time-dependent aspects in the query.

19. The system of claim 12, wherein extracting one or more document chunks from the database further comprises performing at least one of a keyword search and a k-Nearest Neighbor (KNN) search on the plurality of document chunks in the database.

20. A non-transitory computer-readable medium storing computer-executable instructions that, when executed by one or more processors of a computer system, configure the computer system to at least:
receive a query in natural language;
generate an input for a large language model, the input comprising a prompt generated based on the query;
identify a plurality of slots associated with a plurality of sections of a content item;
generate, by the large language model, a query result based on the input, the query result comprising a subset of slots of the plurality of slots selected in accordance with the plurality of sections;
extract one or more document chunks from a database storing a plurality of document chunks as one or more relevant document chunks associated with the query result;
format the one or more relevant document chunks into a response to the query; and
providing the response to the query to a client system,
wherein the plurality of document chunks are generated by dividing each document of a plurality of documents into the plurality of document chunks based on sections within each content item of a plurality of content items.
